(19) United States
(12) Patent Application Publication
(10) Pub. No.: US 2004/0260272 A1
(43) Pub. Date: Dec. 23, 2004

(54) Method and system for intravesicular delivery of therapeutic agents

(76) Inventors: Craig D. Friedman, Westport, CT (US); Peter D. Constantino, Armonk, NY (US); Arindam Datta, Hillsborough, NJ (US); Arthur H. Tinkelenberg, Brooklyn, NY (US)

Correspondence Address: William H. Dippert, Reed Smith LLP, 29th Floor, 599 Lexington Avenue, New York, NY, 10022-7650, US

(21) Appl. No.: 10/692,055

(22) Filed: Oct. 22, 2003

Related U.S. Application Data
(60) Provisional application No. 60/420,180, filed on Oct. 22, 2002.

(51) Int. Cl. A61K 9/22

(52) U.S. Cl. 604/890.1

(57) ABSTRACT

A therapeutic agent delivery implant for implantation into a patient's body comprises a resilient or flexible, at least partially hydrophobic reticulated elastomeric support scaffold; and a hydrophilic coating arranged on said scaffold, wherein said coating contains one or more therapeutic agents for release within the patient. Optionally the coating can contain microspheres or enzymes. In a preferred embodiment, the scaffold comprises a hydrophobic polyurethane, the coating comprises a hydrophilic polyurethane, and the implant has a hemispherical, bullet, football, cylindrical, spherical, or irregular shape. The implant can be delivered through a rigid or flexible delivery instrument that deploys the implant at a desirable site, whereby the implant expands to a size and shape substantially similar to its size and shape before insertion.

### CROSS-REFERENCE TO RELATED APPLICATION

This application is based upon co-pending, commonly assigned, U.S. provisional patent application Ser. No. 60/420,180, filed Oct. 22, 2002, which is incorporated herein in its entirety.

### FIELD OF THE INVENTION

The present invention relates to methods and devices for the intravesicular delivery of therapeutically active agents or materials to the bladder or other privileged mammalian sites for local or systemic use. Preferred embodiments of the invention relate to delivery of therapeutically active substances to the human organs such as the bladder to provide local or systemic therapeutic effects.

### BACKGROUND OF THE INVENTION

Orally ingested drugs are subject to four possible fates in a mammal: First, the drug can be absorbed through the mucosa of the stomach or small intestine and delivered to a vein unaltered to be later metabolized in the liver or other organ to more soluble forms that be utilized by their target organ or metabolized to a form for elimination. Second, the drug can be metabolized in the proximal gastrointestinal tract without further action in the liver. Third, the drug can be metabolized both by the proximal gastrointestinal tract and by the liver. And fourth, the drug can remain unabsorbed or unprocessed in the gastrointestinal tract, to be passed in much the same state as it was swallowed. The nature of the metabolism pathway of a drug is often quite significant since drug metabolites may have very different direct effects and side effects than does the parent drug.

Alternative routes such as subcutaneous or intravenous injection are unattractive because the risk of infection and the pain associated with injections. Additionally, used needles and syringes must be disposed of properly in a biohazard container which can cause clutter for the user. When a patient or relatively untrained person performs injections, the risk of injecting into a blood vessel increases. If some drugs are injected into the bloodstream, too much of that drug is systemically active and can cause serious effects of even death. Injections themselves cause localized trauma, and often the injected substance can cause localized effects at an injection site; therefore, if repeated injections are necessary, the site of injection needs to be varied to prevent too much damage to the one site. However, patients may find this to be uncomfortable since they are traumatizing a new area of their body each day. This discomfort can lead to non-compliance on the part of the patient.

Insertion into a patient's oral cavity or body cavities, such as with anal, vaginal, or urethral suppositories or pessaries, has also been used for drug delivery. The problem with such insertion has generally been getting the desired substance across the mucous membrane and into the bloodstream without damage to the delivery site. Also, time delay and accessibility have been problems. Additionally, many people are uncomfortable talking about inserting items into body cavities, even for therapeutic purposes.

Transdermal drug delivery has been tried over the years. One of the main problems is transporting the substance across the skin layers and into the bloodstream. Chemical carriers such as DMSO have been tried with some limited success. Also, the use of electrical impulse (electrophoresis) and sound waves (sonophoresis) have been used to drive a drug internally. However, many drugs are just molecularly too large to pass through the dermis. Further, many of the drugs used in transdermal drug delivery systems cause skin irritation which increases the risk of non-compliance by the patient.

Bladder cancers are usually treated with a series of infusions, lasting from one to several months, of anti-cancer drugs through the urethra. These infusions take about one to two hours to occur and require a minor operative procedure. The infused chemotherapeutics are then prevented from being released from the bladder for a period of approximately one hour. After the treatment, the bladder is usually significantly irritated.

Chronic urinary tract infections are often hard to treat since they often respond marginally to oral antibiotics. A urinary tract infection can also spread to the blood stream, causing life-threatening septicemia. In patients that are immunologically compromised or paralyzed, due to a spinal cord injury, for example, urinary tract infections are a major problem since oral antibiotics do not function as a prophylactic to the infection. Accordingly, patients exhibiting these conditions are treated with either oral or intravenous antibiotics. Such patients often are subject to catheterization multiple times a day to remove urine from the bladder so that monthly catheterization for replacement of a drug delivery implant would not be unduly burdensome to the patient, especially as paralyzed patients are often desensate. Economic benefits may also accrue attributable to a reduced need for intravenous antibiotics, hospitalization, and invasive procedures to treat urinary tract infections.

Accordingly it would be desirable to provide a drug delivery system which avoids one or more of the drawbacks mentioned above with injection, insertion or transdermal delivery or for the treatment of bladder cancer or urinary tract infections.

### OBJECTS OF THE INVENTION

It is an object of the invention to provide methods and devices for the intravesicular delivery of therapeutic agents or materials to the bladder or other privileged mammalian sites for local or systemic use.

It is also an object of the invention to provide implants for delivery of therapeutic agents or materials to human organs such as the bladder to provide local or systemic therapeutic effects.

It is a further object of the invention to provide an implant for treatment of bladder cancer by intravesicular delivery.

It is yet further object of the invention to provide an implant for delivering therapeutic agents or materials which comprises a resilient or flexible, at least partially hydrophic reticulated elastomeric support scaffold and one or more therapeutically active agents or materials secured to or supported by the scaffold, for release within a patient.

It is yet further object of the invention to provide an implant for delivery of therapeutically active agents or ingredients which comprise a resilient or flexible, at least partially hydropholic reticulated elastomeric support scaffold and a coating arranged on said scaffold, wherein said coating contains one or more therapeutically active agents or materials for release within a patient.

It is a yet further object of the invention to provide a method for delivering an implant with therapeutically active agents or materials to a patient, which comprises the steps of:

(a) collapsing and compressing an implant comprising a resilient or flexible, at least partially hydrophobic reticulated elastomeric support scaffold and one or more therapeutically active agents or materials;

(b) inserting the collapsed and compressed implant into a delivery instrument;

(c) advancing the delivery instrument into a patient;

(d) deploying the implant at a desired site; whereby the implant will recover substantially to its original shape and size after deployment; and

(e) thereafter withdrawing the delivery instrument.

It is a yet further object of the invention to provide a method of treating a urinary tract condition or disease, which comprises the steps of:

(a) compressing and collapsing an implant comprising a resilient or flexible, at least partially hydrophobic reticulated support scaffold and one or more therapeutically active agents or materials;

(b) inserting the collapsed implant into a delivery instrument;

(c) advancing the delivery instrument through the patient's urethra;

(d) deploying the implant at a desired site within the patient's bladder, whereby the implant will recover substantially to its original shape and size after deployment; and

(e) withdrawing the delivery instrument.

These and other objects of the invention will become more apparent from the discussion below.

### SUMMARY OF THE INVENTION

In accordance with the invention, a therapeutic agent implant for implantation to a mammalian site is provided. The implant comprises a resilient or flexible, at least partially hydrophobic reticulated elastomeric support scaffold and one or more therapeutic agents secured to and/or supported by the scaffold for release at the mammalian site. The therapeutic agent delivery implant is insertable into a mammalian bladder or other suitable site via the urethra and is locatable within the bladder. Optionally the implant is out of stimulative contact with the trigone during the normal daily host routine. Preferably, the therapeutic agent delivery implant remains stable and fixed against the mucous membrane of the bladder away from the trigone. Alternatively, it can float clear of the trigone.

In preferred embodiments of the invention, the implants are intended to have varied shapes and may have a cross-sectional area less than, equal to, or greater than the effective cross-sectional area of the bladder, to the extent that they may move within the bladder. In one embodiment of the invention the therapeutic agent delivery implant can be positioned in the dome of the bladder and permit flow of urine through the therapeutic agent delivery implant material. The therapeutic agent delivery implant of this embodiment can optionally be shaped to engage and lodge against the bladder inner wall and may be configured, sized, and prestressed to have a cross-sectional area in excess of the anticipated maximum cross-sectional area of the intended recipient bladder. Preferred shapes include cylindrical, football, bullet, and sphere. Preferably the therapeutic agent delivery implant is biodurable, porous, reticulated, and compressibly elastomeric and demonstrates resilient delivery.

In another embodiment of the invention, a method of delivering an implant to a mammalian site comprises the steps of:

(a) compressing and collapsing and loading into a delivery instrument such as a cannula, trocar, catheter, or any type of minimally invasive rigid or flexible instrument, optionally one incorporating visualization or electromechanics, such as a cystoscope, laproscope, arthroscope, or endoscope, or the like, a resiliently compressible reticulated therapeutic agent delivery implant having an expanded configuration when deployed;

(b) advancing the loaded delivery instrument through a mammalian urethra to access the bladder;

(c) deploying the drug delivery implant through the delivery instrument into the bladder, whereby the implant will recover substantially to its original shape and size after deployment; and

(d) withdrawing the delivery instrument, leaving the drug delivery implant in the bladder.

In yet another embodiment of the invention a therapeutic agent delivery implant is positioned at or adjacent to any desired site within a patient's body. The implant could be delivered in a non-compressed state, but preferably it is compressed and then loaded into a suitable, flexible or rigid, delivery instrument, such as a cannula, trocar, catheter, or any type of minimally invasive rigid or flexible instrument, optionally one incorporating visualization or electromechanics, such as a cystoscope, laproscope, arthroscope, or endoscope, or the like, the distal portion of the delivery instrument is advanced to a position at or adjacent to a target site, such as an organ, and the implant is deployed.

In a further embodiment of the invention, after a sufficient time or a sufficient amount of therapeutic agent delivery or therapy, to recover the therapeutic agent delivery implant the implant is pulled into a flexible or rigid removal instrument, such as a cannula, trocar, catheter, or any type of minimally invasive rigid or flexible instrument, optionally one incorporating visualization or electromechanics, such as a cystoscope, laproscope, arthroscope, or endoscope, or the like, that has been inserted into the patient's body, for example, into the urethra. More specifically, the removal instrument is inserted into the urethra, and the drug delivery implant is removed from the bladder with the removal instrument. The removal instrument optionally includes a fiberoptic device for viewing the drug delivery implant. In addition, a gripping implement may be optionally deployed through the removal instrument to grip the drug delivery implant and draw it into the removal instrument. Further, the implant may have a projection or feature that facilitates gripping by or connection to the removal instrument.

In yet further embodiment of the invention, a therapeutic agent delivery device comprises:

an at least partially hydrophobic, reticulated elastomeric support scaffold and

at least one therapeutic agent secured to or supported by the scaffold or incorporated into a coating that is supported by the scaffold

and is implanted in a patient's body, within the bladder or elsewhere. It can be delivered through or by means of one of the delivery instruments described above, and it can be removed through or by means of one of the removal instruments described above.

### BRIEF DESCRIPTION OF THE DRAWINGS

One or more embodiments of the invention and of making and using the invention, as well as the best mode contemplated of carrying out the invention, are described in detail below, by way of example, with reference to the accompanying drawings, in which:

FIG. 1 is a schematic cross-sectional view of a human bladder;

FIG. 2 is a lateral cross-sectional view of an implant for delivering therapeutic agents according to one embodiment of the invention;

FIG. 3 is an underneath plan view of the therapeutic agent delivery implant shown in FIG. 2;

FIG. 4 is a partial cross-sectional view of a modified embodiment of the therapeutic agent delivery implant shown in FIG. 2 after implantation into the bladder shown in FIG. 1;

FIG. 5 is a lateral elevational view of another embodiment of implant;

FIG. 6 is a lateral elevational view of a second embodiment of a therapeutic agent delivery implant according to the invention;

FIG. 7 is a plan view of a spherical embodiment of a therapeutic agent delivery implant according to the invention;

FIG. 8 is a lateral elevational view of a fusiform or “football” embodiment of therapeutic agent delivery implant according to the invention;

FIG. 9 is a schematic view of the implant of FIG. 8 floating in the bladder;

FIG. 10 is a lateral elevational view of a bullet-shaped embodiment of therapeutic agent delivery implant according to the invention;

FIG. 11 is a lateral elevational view of a cylindrical embodiment of therapeutic agent delivery implant according to the invention;

FIG. 12 is a plan view of a biconcave disc-shaped biologic agent delivery implant according to the invention;

FIG. 13 is cross-sectional view on the line 13-13 of FIG. 12;

FIG. 14 is a lateral elevational view of a spaghetti strand biologic agent delivery implant according to the invention;

FIG. 15 is a lateral elevational view of a biologic agent delivery implant according to the invention having a configuration like that of a mophead;

FIG. 16 is a plan view of one implant introducer instrument having the form of a rigid cystoscope;

FIG. 17 is a plan view of another implant introducer instrument having the form of a flexible cystoscope;

FIG. 18 is a plan view of a further implant introducer instrument having the form of a plunger-equipped catheter;

FIG. 19 is a plan view of a still further implant introducer instrument also in the form of a forceps-equipped catheter;

FIG. 20 is an enlarged partly sectional view of the tip of a modified catheter such as that shown in FIG. 18; and

FIG. 21 is a schematic sectional view of a portion of a human bladder inner wall.

### DETAILED DESCRIPTION OF THE INVENTION

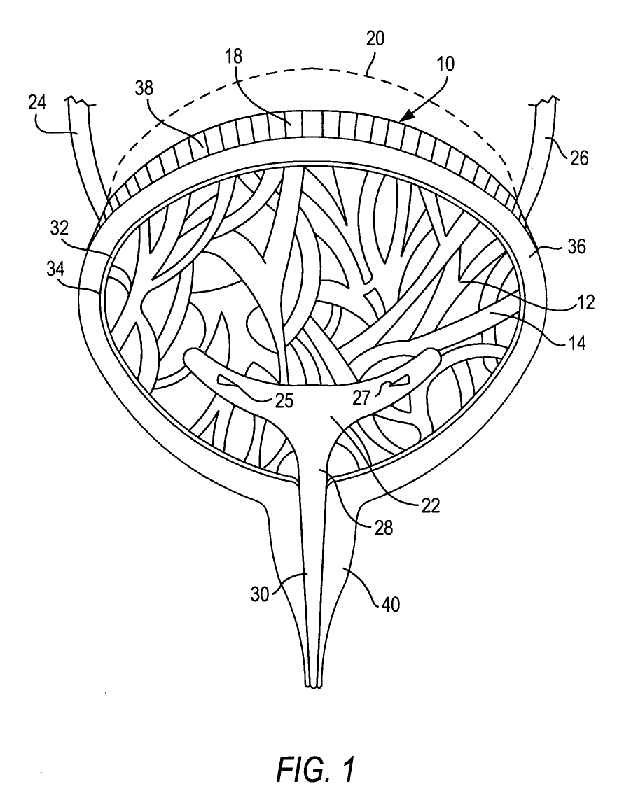

The invention can perhaps be understood better from the drawings. As shown in FIG. 1, the urinary bladder 10 is a hollow, muscular organ located in the pelvic cavity. The common functions of the bladder are accommodation of urine, storage of urine, maintenance of urine composition and facilitation of voiding at appropriate time intervals. When the bladder 10 is empty, the inner walls 12 are retracted into folds 14 defining a relatively small bladder volume. As the bladder 10 fills with urine, it distends, the inner walls 12 extend and become smooth, and the superior surfaces 18 of the inner walls expand upwardly into distended volume, shown in broken lines 20, with the expanded walls defining a relatively larger bladder volume. A typical human bladder has the capacity to hold up to approximately 600 milliliters of urine or, in some cases, as much as about one liter. The desire to micturate or urinate usually occurs when the bladder contains approximately 150 milliliters of urine.

The inner floor of bladder 10 includes a triangular area called the trigone 22, which has openings at each of its three angles. The posterior aspect of the trigone 22 is the base of the triangle where the ureters 24 and 26, bringing urine from the kidneys, empty into bladder 10 through openings 25 and 27 at the two posterior corners of trigone 22. The anterior aspect of the trigone 22 at the apex of the triangle is a funnel-shaped extension called the neck 28 of bladder 10, which opens into the urethra 30. The trigone 22 generally remains fixed in position while bladder 10 is distending and contracting.

The wall of bladder 10 has four layers. An innermost layer called the mucous coat or urothelium 32 has a thickness which changes as bladder 10 expands and contracts, becoming thinner as bladder 10 expands. A second layer is the submucous coat 34 which contains connective tissue and elastic fibers. A third layer is the muscle coat 36 which is made mostly of smooth muscle having fibers interlaced to form what is known as the detrusor muscle 40. The outer layer is the serous layer and is only found on the superior portion or dome 38 of the bladder 10.

The portion of the detrusor muscle 40 that surrounds the neck of the bladder 28 forms the internal urethral sphincter which controls micturition. The internal urethral sphincter sustains a contracted state to prevent the bladder from emptying until the pressure of urine accumulating within the bladder reaches a threshold level. When the threshold level is reached, the parasympathetic nervous system is triggered to intermittently relax the detrusor muscle 40, causing a sensation of urgency. The external urethral sphincter, which is under voluntary control, must be relaxed for micturition to take place.

Many factors can provide the sensation of a full bladder and the need for micturition, including, for example, distention of the bladder, usually due to urine or in some instances gas, irritation of the lining of the bladder and the viscosity of the bladder contents, namely, urine. The bladder may generate a sensation of being full, even when it is not, if abnormally viscous urine is present.

Consequently, any objects, materials, or substances introduced into bladder 10 for therapeutic purposes should avoid inducing any of these conditions or they may trigger unacceptable micturition and may even be voided before they can have their desired therapeutic effects. Additionally, trigone 22 is extremely sensitive to contact with foreign objects so that any object or material introduced into the bladder should also be designed to have minimal or no contact with trigone 22. Further, any implant should preferably be designed to avoid or minimize prolonged contact with the trigone, especially to avoid stimulation or obstruction of urine flow or the bladder neck. In addition, non-selective or selective sympathetic or parasympathetic receptor blocking gents can be administrated, examples of which agents include lidocaine or similar derivatives, capaciacin, and capaciacin-like agents. These agents are intrinsically incorporated either initially or later to induce bladder tolerance to an implant for the duration of the “implant” residence in the bladder. In addition, such an agent can be used to treat a given physiological condition.

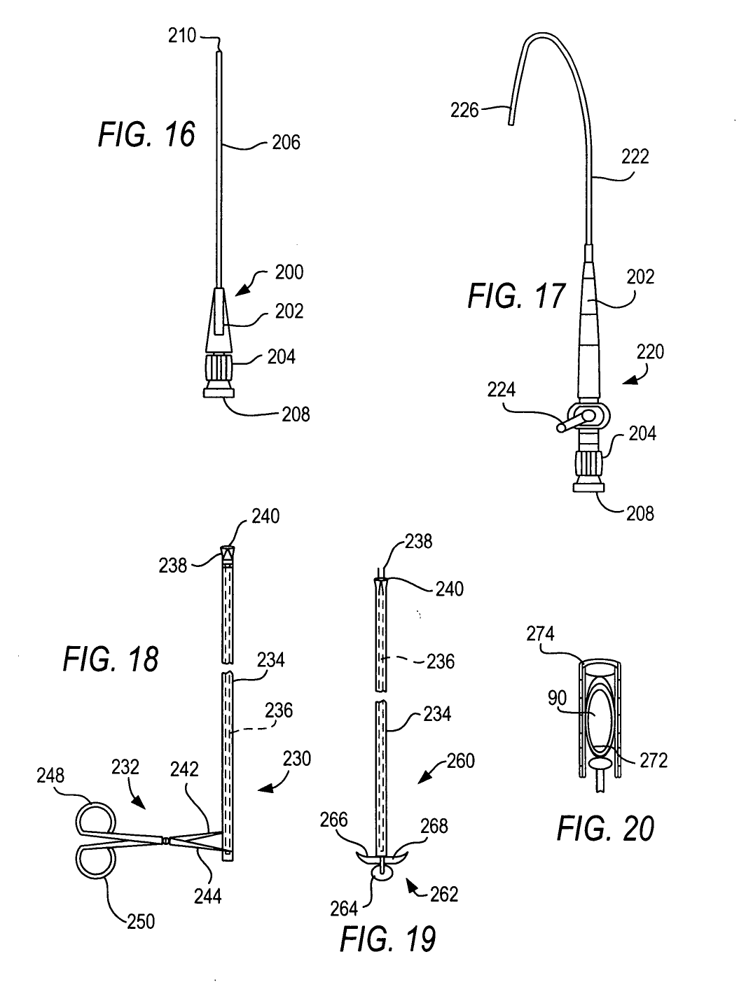

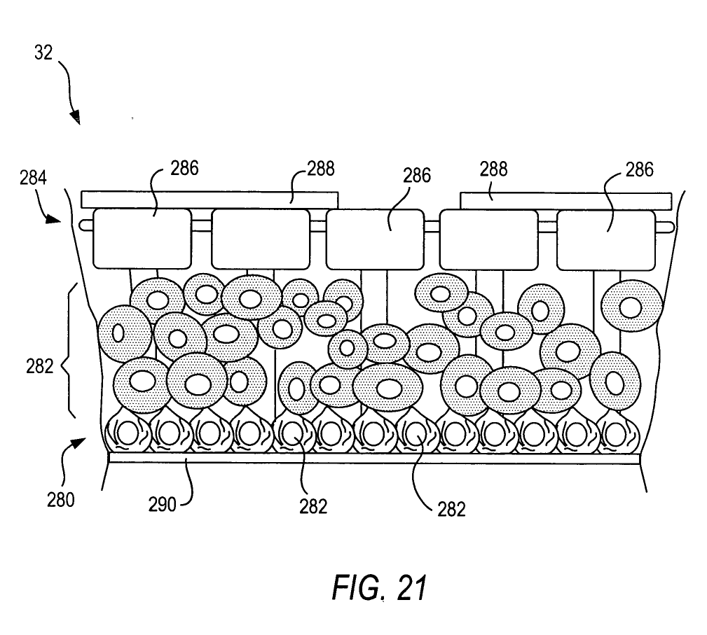

The innermost layer of the bladder wall 12, urothelium 32, functions physiologically in the accommodation and storage of urine, maintenance of urine composition, facilitation of voiding and containment of potential toxins within the bladder to prevent their systemic absorption. The urothelium has three cellular zones: a basal layer, which is the outermost layer with respect to the interior of the bladder and contains cells which are mostly germinal in nature; an intermediate cell layer; and an innermost layer which lines the lumen of bladder 10 and comprises epithelial umbrella cells. The luminal surfaces of the umbrella cells are coated with a layer of glycosaminoglycans. This anatomy is illustrated in more detail in FIG. 16 and may be better understood from the description of that figure set forth below.

The permeability of the bladder wall 12 to chemical and biological substances such as toxins, bacteria and therapeutic agents is believed to be dependent upon the permeability of the urothelium 32. The umbrella cells in the urothelium act as a primary urine-plasma barrier to keep substances within the urine from re-entering the circulatory system, even under extremely high concentration gradients between the plasma and the urine. Some factors affecting bladder wall permeability include: passive diffusion, osmotically driven diffusion, active transport, and the inertness of the membrane to the solutes to which it is exposed. When the device of the invention is inserted into the bladder but is intended to treat conditions or infections within the patient but external to the bladder, such as osteomylitis, it is advantageous to add physiologically acceptable membrane solubilizers such as protemine sulfate or polyethylene gycol to the therapeutic agents or the carriers to cause transient permeability and permissiveness of the mucosa to enter its submucosa.

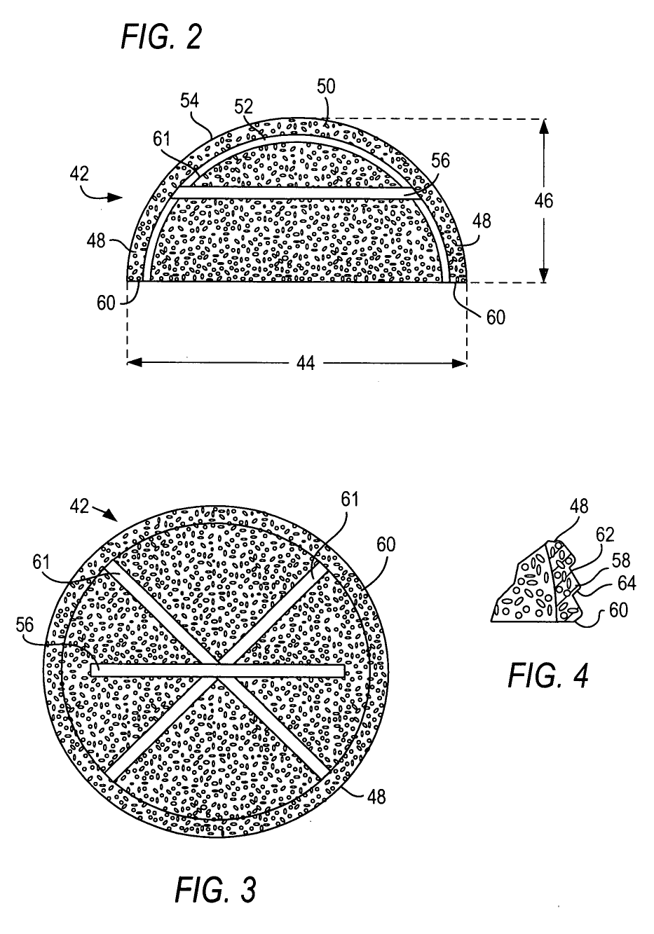

The embodiment of the invention shown in FIGS. 2 to 5 provides an apparatus and method for delivery to privileged mammalian sites such as bladder 10 of therapeutic agents, including not only active pharmaceutical substances such as drugs but agents such as enzymes, antibodies, cells, DNA, RNA, viruses, bacteria, vectors and the like. The term therapeutic agents is used herein to embrace all such therapeutic agents, unless the context clearly indicates otherwise. A more detailed listing of useful therapeutic agents is set forth below.

Implant

The apparatus and method of the invention employ a novel implant for delivering drugs and other biologic agents, as a carrier for a drug or other therapeutic agents. An implant can have any of a wide range of shapes and configurations, according to the particular circumstances of a given application, including cylindrical, football, bullet, spherical, or an irregular shape, as shown below. Illustrated in FIG. 2 is an embodiment of an implant 42 which is suitable for delivery of therapeutic agents into the urinary bladder 10. The delivered one or more therapeutic agents may be for use locally or systemically or may be delivered to the bladder for systemic transport to other in vivo application sites as will be described in more detail hereinafter.

Pursuant to an embodiment of the invention, implant 42 is a novel article or device which comprises a drug-bearing porous, biodurable, reticulated elastomeric matrix designed to be inserted into the bladder through a cannula, catheter, trocar, cystoscope, or other suitable introducer instrument. Preferred embodiments of implant 42 comprise a one-piece flexible thin-walled, shell-like hollow body, such as the superior hemisphere of a plastic ball, which hollow body is collapsible to a compact configuration for accommodation in the introducer instrument and is expansible to an expanded or extended working configuration in situ. To these ends, implant 42 may be fabricated of a resilient or flexible porous material, preferably a resilient and flexible porous material, for example, a resilient foam, especially, for example, a reticulated polyurethane foam coated on its pore surfaces with a drug-bearing material such as a partially hydrophilic foam.

The particular embodiment of implant 42 illustrated in FIG. 2 has a domical, optionally hemispherical, shape, to occupy much or most of the space within bladder dome 38 when bladder 10 is substantially empty, and has a diameter 44 and a height 46. Where the domical shape is a true hemisphere, or approximation thereof, which is a functionally useful shape that is also convenient to manufacture, height 46 is the radius of the sphere and therefore is equal to one half of diameter 44. However, different portions of a sphere, or other shape may be employed, for the domical shape of implant 42, if desired, and in particular, height 46 may be rather less than half of diameter 44, for example, up to about 20 percent less. Such a shallower shape for implant 42 is contemplated as being less likely to contact sensitive trigone 22 when properly oriented.

As shown in FIG. 3, implant 42, being hemispherical, is circular in cross-section. However, many modifications may be made to the particular shape and configuration of implant 42, as will be, or will become, apparent to those skilled in the art, and as are described herein. In particular, the domical shape of implant 42 may have other smoothly curved configurations than part-spherical, and may for example, be a partial ellipsoid or a partial paraboloid.

Articles fabricated of foam and other porous materials may be considered to have both external and internal surfaces. The term external surface is used herein to reference the outer surface of the article itself, while the term internal surface or internal surfaces is used to reference the surfaces of the pores or other openings in the porous material. Thus, for example, a cube of unit length per side, of an open-celled foam having some tens of pores per linear side, has six flat square external surfaces, and a complex, extended array of internal surfaces permeating the whole body of the cube. While the total external surface area will be six units, the internal surface area may be much higher, some tens or even hundreds of units depending upon the porosity and particular microstructure of the foam material. A domical shape, as described above, is a useful shape for implant 42 providing an extended external surface area in the upper portion of a site such as bladder 10, away from the sensitive trigone 22.

Implant 42, as illustrated, has a peripheral sidewall skirt portion 48 and an upper portion 50. Preferably, diameter 44 is selected to be somewhat larger than the largest diameter or girth of the target bladder 10 so that the outer skirt portion 48 is resiliently urged outwardly, preferably with an appropriately gentle force, against bladder 10's inner walls 12, by the resilience of the implant material. Such outward urging can help locate implant 42 at a suitable position or positions within bladder 10, especially a position reducing or minimizing risk of contact with the trigone 22. Thus, for example, skirt portion 48 is preferably positioned to engage and exert a modest outward force against a sidewall portion of inner walls 12, referring to an upright bladder position and a preferred orientation of implant 42. It should be recognized that the implant may be another shape and/or size, as discussed below, and that the implant may float easily within bladder 10.

Height 46 is preferably chosen to enable implant 42 to remain in the above-described preferred position when the bladder 10 contracts to its smallest configuration as it is voided. Preferably height 46 is such that little downward pressure is exerted on the upper portion 50 of implant 42 by bladder dome 38 when the bladder contracts, to avoid displacing implant 42. However, light pressure from bladder dome 38 on implant 42 may be acceptable. To this end, implant 42 may, if desired, be formed of a readily flexible material, at least, in its upper portion 50 to accommodate the contractions of bladder dome 38.

In the domical embodiment illustrated, the external surfaces of implant 42 comprise a concave inner surface 52 and a convex outer surface 54. As shown in FIG. 2, inner surface 52 and outer surface 54 are substantially equidistant from one another throughout their extent, so that implant 42 has a substantially uniform thickness between the two surfaces 52 and 54, subject to manufacturing and microstructural variations. However, such uniformity is merely one embodiment of the invention, and implant 42 may otherwise have uneven thickness.

Preferably the geometry and materials of an implant such as implant 42 are selected to provide an implant which can meet the requirements of being capable of supporting a useful quantity of a therapeutic agent to be delivered; of being collapsible, while bearing the useful quantity of therapeutic agent, into an introducer instrument for implantation to the intended site; of being deployable at the desired site in a manner which permits access of bodily fluids to diffuse the therapeutic agent from the implant and which does not interfere with normal bodily functions; and of being able to substantially recover its shape and size upon deployment. Preferably, when utilized as a urinary bladder implant, an implant such as implant 42, in its deployed configuration, has an extended surface area on which the therapeutic agent or agents are supported for release and does not significantly affect the available urinary volume of the bladder. Preferably, also, an implant such as implant 42 is deployed to release the therapeutic agent in the vicinity of the biological structures that can utilize it or receive it for transport elsewhere, for example, in the vicinity of bladder inner walls 12, especially dome 22 as in the case of implant 42.

A domical configuration of implant 42 such as that illustrated in FIG. 2 is intended to fulfil some or all of these objectives when embodied in a suitable material such as the polymeric foam and other porous materials described herein. In particular, a domical shape, or equivalent cap-like or tent-like shape, which converges upwardly toward a center from an open or preferably closed loop periphery, embodied in a sheet-like porous material, provides a device which can be implanted to body sites such as the urinary bladder by collapsing the implant into a small, longitudinal volume. Such a device can extend, or be extended within the bodily site to have a substantial external surface area for exposure of the material of the implant to, and permeation of the material by, bodily fluids, or possibly gases. Other shapes can accomplish this as well.

The diameter 44 and the height 46 of implant 42 can be varied to provide implants 42 of different sizes to be accommodated within bladders of differing sizes. Alternatively, a single size suitable for insertion into a wide range of different-sized bladders may be employed. Such a universal implant could be sized to the smallest bladder in the range. Another alternative is for the implant to be trimmed to size, at the point of care, by the physician.

Implant 42 can be circular in cross-section, although other, preferably symmetrical, cross-sectional shapes could be employed especially, for example, polygonal shapes such as hexagonal, octagonal, dodecagonal, or the like.

The wall thickness of implant 42 is preferably approximately uniform, although may be varied if desired. For example, skirt portion 48 may have alternating thinner and thicker arcuately extending portions to facilitate packing into the implantation device.

Optionally, implant 42 may include reinforcing structures such as ribs 61 adhered to or molded into implant 42, which ribs 61 may be formed of a non-porous, structural biocompatible polymer, for example, a polyurethane. As shown in FIGS. 2 and 3, implant 42 optionally comprises a pair of cross-like perpendicularly disposed semicircular ribs 61 on inner surface 52 of implant 42. Preferably, ribs 61 are sufficiently flexible to bend to be accommodated in a delivery instrument and are lightly prestressed into the arcuate configuration shown in FIG. 2. Ribs 61 can have a partial extent, for example, stopping short of the center of the cross configuration. Such ribs can be employed in any desired configuration to help the implants of the invention adopt a desired configuration in situ. For example, in an alternative configuration, ribs 61 could comprise rings, or arcs extending around the interior, or exterior, of implant 42 approximately parallel with surface 60. However, ribs that can adopt a mostly straight line configuration in a compressed configuration of implant 42 are preferred.

FIG. 4 illustrates another embodiment where the thickness of implant 42 is varied in a useful manner. In the FIG. 4 embodiment, skirt portion 48 is provided with a number of peripheral ridges 58 to engage bladder inner walls 12. Ridges 58 extend preferably continuously around skirt portion 48 parallel to the lower periphery 60 of implant 42, in a circumferential manner in the case of a hemispherically shaped implant 42. Alternatively, ridges 58 may be discontinuous with significant gaps between one ridge portion and the next. As shown, ridges 58 have an asymmetric sawtooth profile with an upper more gently sloped land 62 and a lower more steeply sloped land 64, referring to the orientation of implant 42 shown in FIG. 2, which corresponds approximately to the orientation preferred in an upright bladder 10. When employed with a resilient implant 42 having a diameter 44 slightly greater than the relevant cross-section of bladder 10, whereby implant 42 is urged outwardly into engagement with bladder inner walls 12, the asymmetry of ridges 58 gives them a cam-like action tending to urge implant 42 upwardly in bladder 10, as bladder 10 repeatedly contracts during urination. Furthermore, lower lands 64 tend to hold implant 42 approximately in place, once it is suitably positioned, restraining it from encountering trigone 22.

While the illustrated embodiment of implant 42 is of continuous, monolithic, one-piece construction, it will be understood that other constructions may be employed. For example, implant 42 may be formed with a number of uniformly distributed or localized relatively large pore openings. Rather than being of monolithic construction, implant 42 may comprise multiple segments for example, from to 500, or from 10 to 100, adhered or otherwise secured together, portions of disparate materials interspersed together to form a coherent whole, or may be of laminar construction with two or more layers adhered together of materials of differing characteristics. Thus, implant 42 could comprise a relatively larger pored radially outer layer and a relatively smaller pored radially inner layer to deliver drug preferentially into the urine adjacent the bladder walls 12 rather than to the interior and lower portions of the bladder where the drug will be voided during urination. Controlled, or limited retention of urine between implant 42 and the bladder inner walls 12, promoted by engagement of skirt portion 48 with bladder inner walls 12 can also help control drug losses attributable to urination.

The therapeutic agent delivery implant can also contain a radiopaque or sonically reflective substance for viewability of the implant by radiography or ultrasound to determine the orientation, location and other features of the implant 42.

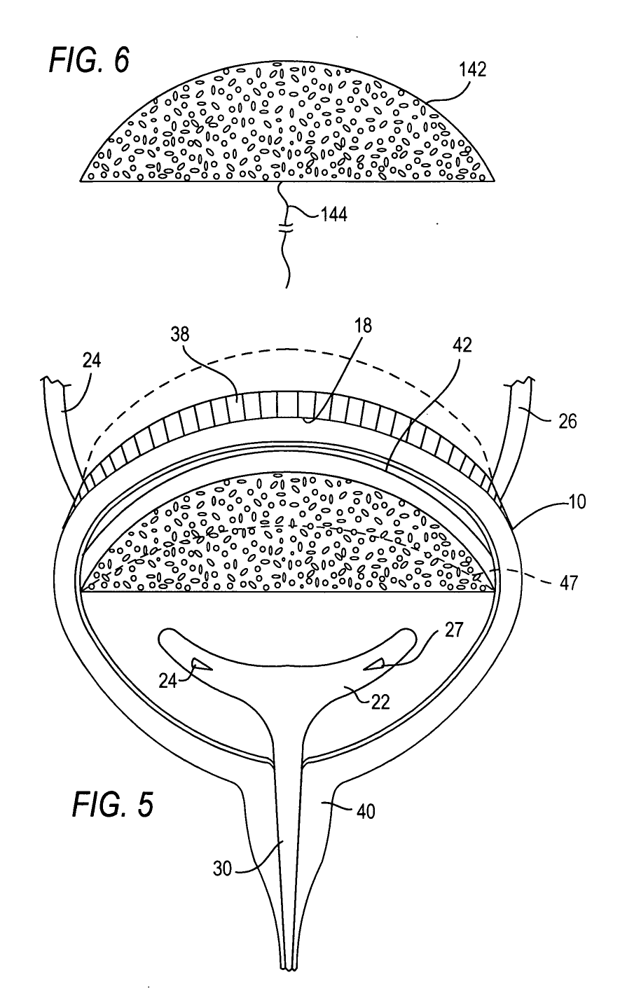

As is illustrated in FIG. 5, implant 42 may tend to reside in the dome of bladder 10. As bladder 10 expands due to filling and contracts due to micturition, the implant 42 can be resiliently compressed and relaxed, if necessary, by the bladder walls 12 so as to be retained in the vicinity of bladder dome 38, clear of the sensitive trigone 22.

As is also illustrated in FIG. 5, device 46 can be a solid domical shape with a lower surface as is illustrated by broken line 47. This configuration provides a more substantial implant device having more mass and, when constructed out of foam, substantial pore surface area that can support more of a biologically active substance than a shell-like configuration such as that shown in FIG. 5.

As illustrated in FIG. 6, a modified therapeutic agent delivery implant 70 additionally may optionally have a centrally attached cord 72 or other pendant flexible, cord-like structure, which can be readily gripped, to facilitate removal of implant 70 from bladder 10, for example by a forceps inserted through a cystoscope, or other suitable instrument. Implant 70 is formed of a thin, flexible material so that it can invert as it is drawn into the cystoscope or other introducer instrument. If desired, cord 72 can be sufficiently long to extend from implant 42, into the urethra 30, or even long enough to extend through the urethra 30, and to project externally. Cord 72 is an example of the gripping member that may be attached to or part of any implant of any shape or size according to the invention.

In one embodiment of the invention, implant 42 can include a loop 56, tab or other grippable structure to facilitate retrieval of implant 42 from a site of implantation. Loop 56 can, for example, comprise a single piece of flexible material extending between opposed sides of skirt portion 48 beneath upper portion 50. Alternatively, it could be Y-shaped or cross-shaped, being secured to skirt portion 48 at three or four spaced apart locations. Loop 56 is preferably formed of relatively high tensile strength non-porous, polymeric material, although it could be formed of the same material as the body of implant 42. Loop 56 is intended to be gripped by a forceps inserted through an introducer instrument.

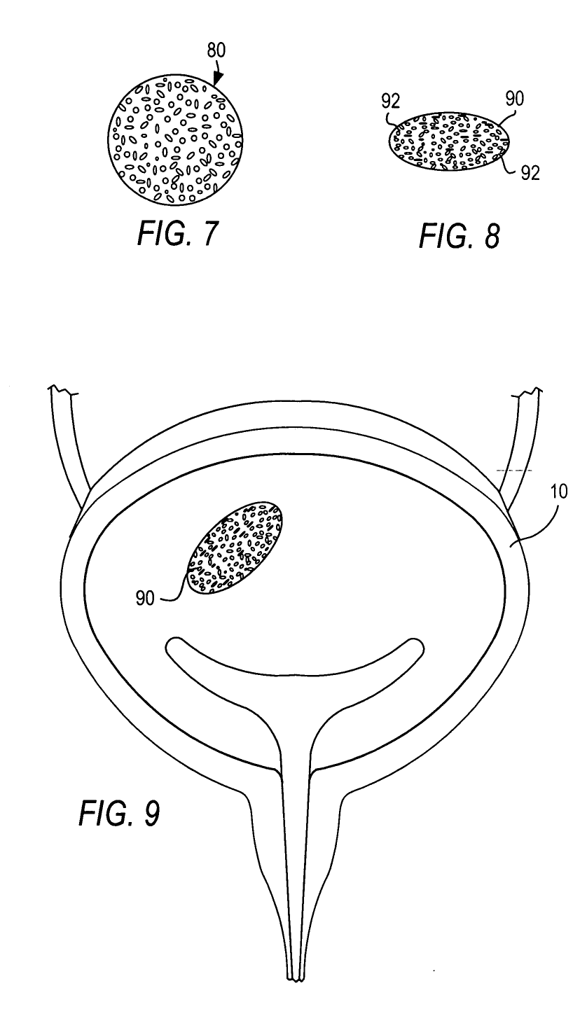

Some additional possible embodiments of novel drug delivery implants pursuant to the invention are illustrated in FIGS. 7 to 13. Other embodiments will be, or will become, apparent to those skilled in the art. These implants can with advantage all be constructed in one piece from a resiliently compressible, spongy foam composite capable of releasably supporting useful quantities of a useful therapeutic agent on its pore surfaces, or may be constructed from other suitable materials, as described herein.

As shown in FIG. 7, an implant 80 has an approximately spherical shape and is sized to be readily accommodated within the bladder, being for example, from about 1 to about 10 cm in diameter, preferably from about 2 to about 6 cm in diameter. A particularly preferred diameter is a maximal size providing a sphere which can just be accommodated within the minimum normal bladder volume, without significant compression of the implant 80.

Implant 80 is solid in the sense that the whole volume of the implant is filled by foam or other suitable implant material, in contrast to the relatively thin-wall, shell-like construction of implant 42 which has a hemispherical outer periphery and a hemispherical hollow interior. However, this solid material volume of implant 80 includes a myriad of small internal interconnected hollow pore spaces, which are accessible by external fluids, such as body fluids in situ, to provide an extended surface area which with a suitable surface coating can be employed for drug release.

The implant 90 illustrated in FIG. 8 has a fusiform or ellipsoidal shape, much like a football having rounded ends 92 and cross-sections perpendicular to the paper, along the length between ends 92, which are approximately, or generally, circular. The maximum length of implant 90 between ends 92, that can be readily accommodated in a given bladder 10, may be a little greater than the equivalent maximal sphere 80 for the same bladder 10 and the maximum cross-sectional diameter may be a little less. As shown in FIG. 9, fusiform implant 90 can float relatively freely within bladder 10 with no particular orientation being required.

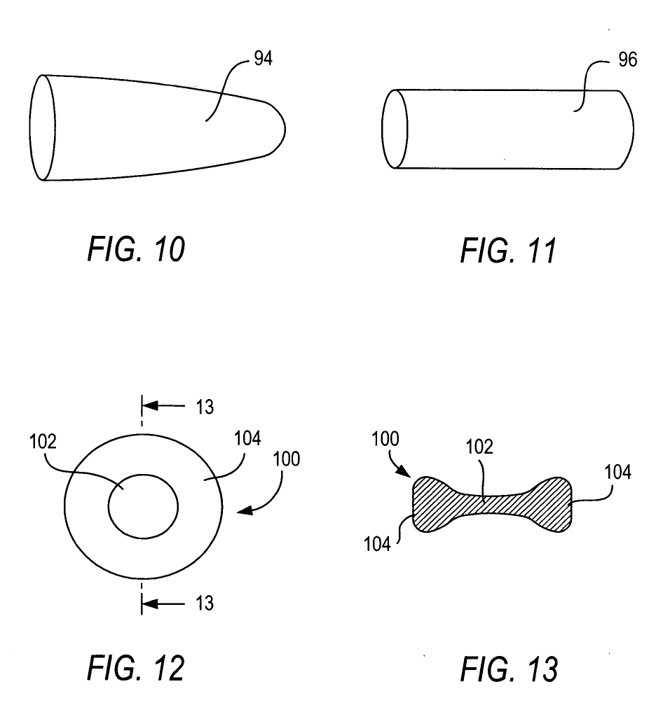

An implant 94 in FIG. 10 has a bullet-like shape, and an implant 96 in FIG. 11 has a cylindrical shape. Other suitable solid implant configurations (not shown) include cubic, elongated cuboid, trapezoidal, parallelepiped, ellipsoid, fusiform, rod, tube, sleeve, elongated prismatic form, or a folded, coiled, helical or, other more compact configuration irregular, and other solid shapes having more or less flat surfaces. Some elongation of the shape is advantageous for compression for implantation. In another embodiment, the elastomeric matrix or the scaffold having such a form has a diameter or other maximum dimension from about 2 cm to about 10 cm.

The longer and thinner shape of implant 90, 94, or 96 as compared with, for example, a sphere, renders implant 90, 94, or 96 particularly suitable to be laterally compressed to fit into an introducer instrument. As shown in FIG. 20 below, an implant such as implant 42, 90, 94, or 96 can readily be compressed into the small pencil-like object and fitted into the cylindrical end portion of an introducer catheter or the like, where a plunger enables the compressed implant to be discharged from the catheter or the like at the desired site of implantation, for example, bladder 10.

Implants 80, 90, 94, 96 and the other solid implants described are useful, space-occupying, free-floating, preferably buoyant, implants which have the following advantages:

ease of fabrication of relatively simple shapes;

ease of loading into an introducer cannula, trocar, catheter, or any type of minimally invasive rigid or flexible instrument, optionally one incorporating visualization or electromechanics, such as a cystoscope, laproscope, arthroscope, or endoscope, or the like;

a large therapeutic agent-bearing volume of implant for a given bladder size; and

ease of retrievability because an end or any other portion of the implant 80, 90, 94, or 96 can be gripped by a cannula-inserted forceps enabling the implant to be withdrawn into the cannula, and be compressed to fit the cannula as the forceps is retracted.

A large implant volume of appropriate porosity provides a large internal surface area for contacting drug-bearing materials with urine. Use of a highly porous implant material having a low bulk density assures that the urine capacity of bladder 10 is not unacceptably impacted because a major proportion of the volume of the implant can be occupied by urine, as will be apparent from the physical properties of the implant material. Consistent with what is shown in FIG. 9, such solid implants can float relatively freely within bladder 10 with no particular orientation being required.

The implants shown in at least FIGS. 7 to 11 have relatively high volume to external surface area ratios. Such ratios make such implants well-suited to relatively long term delivery of therapeutic agents or other active ingredients at relatively low to moderate dosage rates.

Solid shape implants, for example, those of FIGS. 7 to 11, are relatively unlikely to contact the trigone or block the ureter openings 25, 27. However, even should the implants locate themselves in such a position, use of a porous implant material will ensure that urine flow is not blocked. Use of a flexible, resilient implant material can ameliorate the response of trigone 22 to contact. If desired, a relatively soft implant material may be employed, or the outer surface of any of the novel implants described herein can be coated with a soft material for example, a hydrophilic polyurethane layer which may be additional to any internal pore coating layer. Any such protective layer should be applied so as to permit liberal fluid access to the interior of the implant, for example, by applying such a layer only to the more prominent surfaces of the implant 42, 80, 90, 94, or 96 that may encounter trigone 22, for example, to the ends 92 of implant 90 or to the lower peripheral surface 60 of implant 42.

Other suitable solid shapes providing some or all of the above-described advantages will be or become apparent to those skilled in the art.

The embodiment of implant 100 shown in FIGS. 12 and 13 has the shape of a biconcave disc, much like a contraceptive diaphragm or red blood cell. Implant 100 comprises a relatively thin central disc 102 and a thickened circumferential rim 104. Rim 104 provides storage volume for biological actives adjacent inner walls 12 of bladder 10 or other biological structure. The thinner disk portion 102 facilitates compression in to a shape that will fit within an introducer instrument. Implant 100 can optionally be buoyant and be free floating within bladder 10, and sized to be a relatively close fit into the dome of bladder 10. When suitably sized to a particular bladder 10 and placed in the dome of the bladder, the biconcave shape may be retained in place as bladder 10 contracts on device 100.

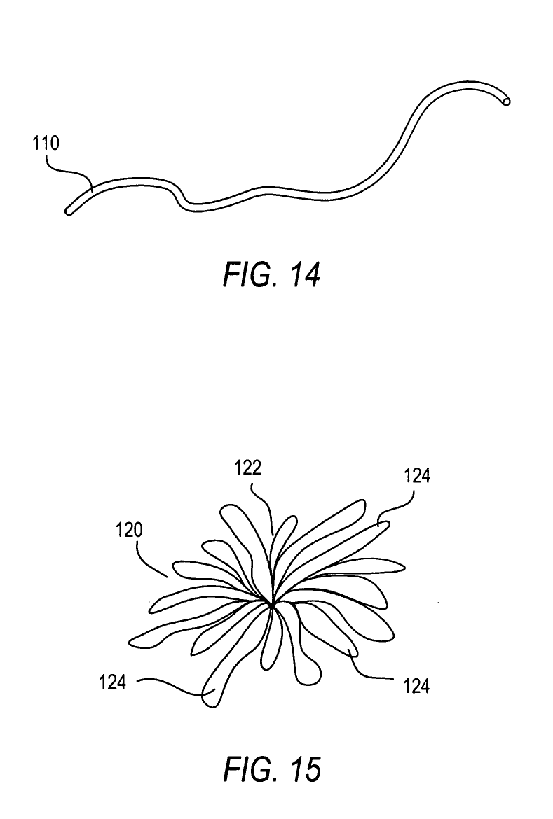

The spaghetti strand implant 110 shown in FIG. 14 has a configuration resembling a piece of cooked spaghetti, linguini or other such pasta and comprises a single long flexible piece of foam or other suitable porous or extended surface area material, as described herein. Spaghetti strand implant 110 may have any desired cross-sectional shape such as square, circular or flattened to give the implant a ribbon-like configuration. Alternatively, implant 110 may be tubular, having an annular cross-sectional shape. Though shown as ended, spaghetti strand implant 110 may comprise an endless loop. While a uniform cross-section throughout the length of implant 110 is convenient, it is not necessary.

Spaghetti strand implant 110 can have any suitable dimensions, for example, a length of from about 0.5 to about 50 cm, preferably from about 2 to about 25 cm, more preferably from about 5 to about 10 cm. Spaghetti strand implant 110 can have any suitable average cross-sectional area, for example from about 0.0025 cm² to about 1 cm², preferably from about 0.01 cm² to about 0.25 cm². The length can be from about 2 times to about 100 times the average width of the strand, preferably from about 5 times to about 20 times the average width.

Spaghetti strand implant 110 may be folded and compressed to fit into an introducer instrument and is easily withdrawn by gripping with a forceps, preferably in a central region of the implant 110. Spaghetti strand implant 110 has modest mass can be fabricated to have a density close to that of urine, or a little less for buoyancy and will accordingly have little irritant effect if it should contact the urine.

Spaghetti strand implant 110 has the advantages of easy insertion and removal via a cannula or other removal instrument and of having a large external surface area relatively to its volume.

The implant 120 shown in FIG. 15 can be described as a mophead implant and has a head portion 122 from which project strands 124 of foam or other suitable material. Strands 124 may be similar to spaghetti strand 110 illustrated in FIG. 9. The configuration of mophead implant 120 provides a very large external surface area for contact with the urine. As with implant 90 shown in FIG. 9, mophead implant 120 can float relatively freely within bladder 10 with no particular orientation being required.

Mophead implant 120 also has the advantages of easy insertion and removal via a cannula and of having a large external surface area relatively to its volume. Depending upon the particular characteristics of the release mechanisms employed spaghetti strand implant 110 and mophead implant 120 are both suitable for delivering high dosages of drugs over relatively short intervals.

Another embodiment of implant (not shown) comprises multiple spaghetti strand pieces of implant material assembled, or intertwined together into a ball from which the strand ends may project, analogously to a ball of spaghetti. The strands may be woven, tied. stitched, adhered or otherwise secured together. As an alternative to foam, the strands may be constituted by a woven or nonwoven porous fabric or other such material to which a desired biologic agent is secured, as described herein.

If desired, multiple suitably sized implants can reside in bladder 10, or another implantation site, simultaneously. Different implants bearing different therapeutic agents or therapeutic agent formulations designed to serve separate, non-interfering ends or to work co-operatively may be simultaneously resident in the site of implantation. Spaghetti strand implant 110 is particularly well suited to this purpose.

Expansion in situ, especially in the bladder after delivery in a compressed state through the urethra, can be effected by the inherent recoverable nature of the material of the implants, arising out of resilient structural components of the implants. Alternatively, a suitable expansion mechanism, for example, an umbrella-like lever and spoke mechanism, may be associated with or built into an implant, such that it is maniputable through a cannula, trocar, catheter, or any type of minimally invasive rigid or flexible instrument, optionally one incorporating visualization or electromechanics, such as a cystoscope, laproscope, arthroscope, or endoscope, or the like.

As stated above, the basic therapeutic agent delivery device of the invention comprises a reticulated, at least partially hydrophobic foam scaffold with at least one therapeutic agent carried or absorbed thereon, preferably in a hydrophilic coating. Such coated foam scaffolds are referred to as foam composites, and some of the benefits of the inventive implants and implant systems employing useful physical characteristics of composite or coated or treated foams are as follows:

Agent Binding. A capacity to adsorb or covalently bond chemicals or therapeutically active agents to the hydrophilic polyurethane layer.

Particle embedment. A capability to embed time-release microspheres or other micropackages or particles within the hydrophilic layer, which embedded entities are distributed in three-dimensional space held in place, relative to one another, and are supported, by the hydrophobic scaffold on which the hydrophilic layer is coated.

Controlled release. The binding and particle embedment capabilities can be utilized to provided an implant system for sustained release of specific therapeutic agents in a controlled and defined fashion, that is, in a certain manner affecting either the location or the timing of the release. Controlled release techniques have particular advantages in the context of administering therapeutic agents. For example, the release rate of a therapeutic agent can be predicted and designed for an extended duration; this eliminates problems associated with patients neglecting to take required medication in specified dosages at specified times. Many therapeutic agents have short half-lives. Trapping these therapeutic agents in polymeric matrices increases the time in which the therapeutic agent maintains its activity. Further, the site specific localization of a therapeutic agent achieved with a targeted delivery technique reduces or eliminates systemic side effects that certain medications cause when administered orally or intravenously in large doses.

Compressive elasticity. The compressive elasticity of a foam composite material useful according to the invention is valuable in enabling an implant to be loaded within a cannula, trocar, catheter, or any type of minimally invasive rigid or flexible instrument, optionally one incorporating visualization or electromechanics, such as a cystoscope, laproscope, arthroscope, or endoscope, or the like, for extended periods of time without compromising the ability of the foam to expand to an uncompressed configuration, for example approximately to its original dimensions. Implant expansion from a compressed state enables the implant to occupy space and allow urine or other body fluid flow to permeate the foam throughout the occupied space, enabling actives located anywhere in the foam to diffuse into the body fluid. Implant expansion from the compressed state, also may also enable domical and other suitably shaped implants to fix themselves into position, in vivo, on a short-term or long-term basis.

Fluid Permeability. A preferred foam composite useful according to the invention is a reticulated polyurethane scaffold, which allows for substantial fluid flow-through, or permeability, permitting active drugs and compounds to be carried away from within the implant in the ambient fluid flow. Fluid permeability facilitates membrane transport of therapeutically active substances from the scaffold, coating on the scaffold, or microspheres in the coating and delivery of the therapeutically active substances externally of the implant, for example, to the transitional mucous membrane of the bladder. The continual filling and emptying of the bladder facilitates movement of urine through the implant and leaching of actives.

Tensile Strength. The useful foam composite material can be fabricated with excellent tensile strengthen allowing an implant to be grasped or retrieved with a hook or forceps and withdrawn into a suitable instrument such as a trocar or cannula for removal from the implantation site, e.g., the bladder. Such grasping or hooking action could disrupt or tear less robust materials such as conventional hydrophilic polyurethane. Implants constructed from useful foam composite materials employing a reticulated hydrophobic polyurethane as a substrate or scaffold material, provide an implant that can retain its structural integrity and be removed without undue difficulty.

Scaffold

The implant of this invention or the hydrophobic scaffold is a porous reticulated polymeric matrix formed of a biodurable polymer that is resiliently-compressible so as to regain its shape after delivery to a biological site. The structure, morphology, and properties of the elastomeric matrices of this invention can be engineered or tailored over a wide range of performance by varying the starting materials and/or the processing conditions for different functional or therapeutic uses.

The porous biodurable elastomeric matrix is considered to be reticulated because its microstructure or the interior structure comprises inter-connected open pores bounded by configuration of the struts and intersections that constitute the solid structure. The continuous interconnected void phase is the principle feature of a reticulated structure.

Preferred scaffold materials for the implants have a porous and reticulated structure with sufficient and required liquid permeability and thus are selected to permit urine, or other appropriate bodily fluids, to access interior drug-bearing surfaces of the implants during the intended period of implantation. This happens due to the presence of inter-connected, reticulated open pores that form fluid passageways or fluid permeability providing fluid access all through and to the interior of the matrix for elution of pharmaceutically-active agents, e.g., a drug, or other therapeutically useful materials. Such materials may optionally be secured to the interior surfaces of elastomeric matrix directly or through a coating. In one embodiment of the invention the controllable characteristics of the implants are selected to promote a constant rate of therapeutic agent release during the intended period of implantation. Also, the passageways may be adjusted sufficiently to permit

Any of a variety of materials meeting the foregoing requirements may be employed. A preferred foam is a compressible, lightweight material, chosen for its structural stability in situ, its ability to support the drug to be delivered, for high liquid permeability, and for an ability to substantially recover pre-compression shape and size within the bladder to provide, when loaded with appropriate substances, a reservoir of therapeutic agents that can be released into the urine in the bladder. Suitable materials are further described hereinbelow.

Preferred foams or the hydrophobic reticulated and porous polymeric matrix materials for fabricating implants according to the invention are flexible and resilient in recovery, so that the implants are also compressible materials enabling the implants to be compressed and, once the compressive force is released, to then recover to, or toward, substantially their original size and shape. For example, an implant can be compressed from a relaxed configuration or a size and shape to a compressed size and shape under ambient conditions, e.g., at 25° C. to fit into the introducer instrument for insertion into the bladder or other suitable internal body site for in vivo delivery. Alternatively, an implant may be supplied to the medical practitioner performing the implantation operation, in a compressed configuration, for example, contained in a package, preferably a sterile package. The resiliency of the elastomeric matrix that is used to fabricate the implant causes it to recover to a working size and configuration in situ, at the implantation site, after being released from its compressed state within the introducer instrument. The working size and shape or configuration can be substantially similar to its original size and shape after the in situ recovery.

Preferred scaffolds are reticulated, interconnected pores polymeric materials, having sufficient structural integrity and durability to endure the intended biological environment, for the intended period of implantation. For structure and durability, at least partially hydrophobic polymeric scaffold materials are preferred although other materials may be employed if they meet the requirements described herein. Materials are preferably elastomeric in that they can be compressed easily and resiliently recover to substantially the pre-compression state. Alternative porous polymeric materials that permit biological fluids to have ready access throughout the interior of an implant may be employed, for example, woven or nonwoven fabrics or networked composites of microstructural elements of various forms.

The partially hydrophobic scaffold is preferably constructed of a material selected to be sufficiently biodurable, for the intended period of implantation that the implant will not to lose its structural integrity during the implantation time in a biological environment. The biodurable elastomeric matrices forming the scaffold do not exhibit significant symptoms of breakdown, degradation, erosion or significant deterioration of mechanical properties relevant to their use when exposed to biological environments and/or bodily stresses for periods of time commensurate with the use of the implantable device such as controlled release or elution of therapeutic agents and/or pharmaceutically-active agents, e.g., a drug, or other biologically useful materials over a period of time. In one embodiment, the desired period of exposure is to be understood to be at least 29 days. This measure is intended to avoid scaffold materials that may decompose or degrade into fragments for example, fragments that could move into the neck of the bladder, and possibly block the urethra or cause similar blockages elsewhere in a patient's body or cause unwanted tissue response.

The void phase, preferably continuous and interconnected, of the a porous reticulated polymeric matrix that is used to fabricate the implant of this invention may comprise as little as 50% by volume of the elastomeric matrix, as compared to the volume provided by the interstitial spaces of elastomeric matrix before any optional interior pore surface coating or layering is applied. In one embodiment, the volume of the void phase as just defined, is from about 70% to about 99% of the volume of the elastomeric matrix. In another embodiment, the volume of the void phase is from about 80% to about 98% of the volume of elastomeric matrix. In another embodiment, the volume of the void phase is from about 90% to about 98% of the volume of elastomeric matrix.

As used herein, when a pore is spherical or substantially spherical, its largest transverse dimension is equivalent to the diameter of the pore. When a pore is non-spherical, for example, ellipsoidal or tetrahedral, its largest transverse dimension is equivalent to the greatest distance within the pore from one pore surface to another, e.g., the major axis length for an ellipsoidal pore or the length of the longest side for a tetrahedral pore. For those skilled in the art, one can routinely estimate the pore frequency from the average cell diameter in microns.

In one embodiment of the invention, the porous reticulated polymeric matrix that is used to fabricate the implant of this invention to provide adequate fluid permeability, the average diameter or other largest transverse dimension of pores is from about 50 μm to about 2000 μm (i.e., from about 300 to about 10 pores per linear inch), preferably from about 50 μm to about 800 μm (i.e., from about 300 to about 25 pores per linear inch), more preferably from about 100 μm to about 500 μm (i.e, from about 150 to about 35 pores per linear inch), and most preferably between about 200 μm and about 400 μm (i.e., between about 80 and 40 pores per linear inch.).

In another embodiment of the invention, elastomeric matrices that are used to fabricate the scaffold part of this invention have sufficient resilience to allow substantial recovery, e.g., to at least about 50% of the size of the relaxed configuration in at least one dimension, after being compressed for implantation in the human body, for example, a low compression set, e.g., at 25° C. or 37° C., and sufficient strength and flow-through for the matrix to be used for controlled release of therapeutic and/or pharmaceutically-active agents, such as a drug, and for other medical applications. In another embodiment, elastomeric matrices of the invention have sufficient resilience to allow recovery to at least about 60% of the size of the relaxed configuration in at least one dimension after being compressed for implantation in the human body. In another embodiment of the invention, elastomeric matrices of the invention have sufficient resilience to allow recovery to at least about 90% of the size of the relaxed configuration in at least one dimension after being compressed for implantation in the human body.

In another embodiment of the invention, the porous reticulated polymeric matrix that is used to fabricate the implants of this invention has any suitable bulk density, also known as specific gravity, consistent with its other properties. For example, in one embodiment of the invention, the bulk density may be from about 0.005 to about 0.15 g/cc (from about 0.31 to about 9.4 lb/ft³), preferably from about 0.015 to about 0.115 g/cc (from about 0.93 to about 7.2 lb/ft³) and most preferably from about 0.024 to about 0.104 g/cc (from about 1.5 to about 6.5 lb/ft³).

The reticulated elastomeric matrix has sufficient tensile strength such that it can withstand normal manual or mechanical handling during its intended application and during post-processing steps that may be required or desired without tearing, breaking, crumbling, fragmenting, or otherwise disintegrating, shedding pieces or particles, or otherwise losing its structural integrity. The tensile strength of the starting material(s) should not be so high as to interfere with the fabrication or other processing of elastomeric matrix. Thus, for example, in one embodiment of the invention, the porous reticulated polymeric matrix that is used to fabricate the implants of this invention may have a tensile strength of from about 700 to about 52,500 kg/m² (from about 1 to about 75 psi). In another embodiment of the invention, elastomeric matrix may have a tensile strength of from about 700 to about 21,000 kg/m² (from about 1 to about 30 psi). Sufficient ultimate tensile elongation is also desirable. For example, in another embodiment of the invention, a reticulated elastomeric matrix has an ultimate tensile elongation of at least about 100% to at least about 500%.

In one embodiment of the invention, reticulated elastomeric matrix that is used to fabricate the implants of this invention has a compressive strength of from about 700 to about 140,000 kg/m² (from about 1 to about 200 psi) at 50% compression strain. In another embodiment, reticulated elastomeric matrix has a compressive strength of from about 7,000 to about 210,000 kg/m² (from about 10 to about 300 psi) at 75% compression strain.

In another embodiment of the invention, reticulated elastomeric matrix that is used to fabricate the implants of this invention has a compression set, when compressed to 50% of its thickness at about 25° C., of not more than about 30%. In another embodiment of the invention, elastomeric matrix has a compression set of not more than about 20%. In another embodiment of the invention, elastomeric matrix has a compression set of not more than, about 10%. In another embodiment of the invention, the elastomeric matrix has a compression set of not more than about 5%.

In another embodiment of the invention, the reticulated elastomeric matrix that is used to fabricate the implants of this invention has a tear strength of from about 0.18 to about 1.78 kg/linear cm (from about 1 to about 10 lbs/linear inch).

In a preferred embodiment of a composite foam for use in the practice of the present invention, the foam composite comprises a polyether polyol or polyether polysiloxane based hydrophilic polyurethane coated on the pore surfaces of a hydrophobic polyurethane foam scaffold. Preferred composite foams can have a density of from about 0.03 g/cc to about 0.10 g/cc and a weight ratio of open cell hydrophilic polyurethane coating to the weight of the hydrophobic foam is from about 0.01 to about 15, and preferably from about 0.5 to about 10.

In another embodiment of the invention the reticulated elastomeric matrix that is used to fabricate the implant can be readily permeable to liquids, permitting flow of liquids, including urine, through the composite device of the invention. The water permeability of the reticulated elastomeric matrix is from about 25 l/min./psi/cm² to about 1000 l/min./psi/cm², preferably from about 100 l/min./psi/cm² to about 600 l/min./psi/cm².

The implant of the invention device allows for the control of the flow rate of liquid through device by adjustment of several characteristics. Firstly, the pore-size of pores of carrier, rather support may be adjusted. For example, in the case of the preferred composite foam, the open-pore structure can be produced in a range of precisely controlled pore sizes that contain void volumes of up to 98%. Various pore sizes, typically from about 35 to about 150 pores per linear inch (ppi), enable the use of the hydrophobic polyurethane in specific applications. The high porosity of material helps control permeability and adds to design flexibility.

In general, a suitable, porous, biodurable, reticulated, elastomeric, partially hydrophobic polymeric matrix that is used to fabricate an implant of the invention or for use as scaffold material for the implant in the practice of the present invention, in one embodiment sufficiently well characterized, comprises one of the elastomers that have or can be formulated with the desirable mechanical properties described in the present specification and have a chemistry favorable to biodurability such that they provide a reasonable expectation of adequate biodurability.

Various reticulated hydrophobic polyurethane foams are suitable for this purpose. In one embodiment, structural materials for the inventive porous elastomers are synthetic polymers, especially, but not exclusively, elastomeric polymers that are resistant to biological degradation, for example, polycarbonate polyurethanes, polyether polyurethanes, polysiloxanes, and the like. Such elastomers are generally hydrophobic but, pursuant to the invention, may be treated to have surfaces that are less hydrophobic or somewhat hydrophilic. In another embodiment of the invention, such elastomers may be produced with surfaces that are less hydrophobic or somewhat hydrophilic.

The invention provides a porous biodurable reticulatable elastomeric partially hydrophobic polymeric scaffold material for fabricating an implant or a material. More particularly, in one embodiment, the invention provides a biodurable elastomeric polyurethane matrix which comprises a polycarbonate polyol component and an isocyanate component by polymerization, crosslinking and foaming, thereby forming pores, followed by reticulation of the foam to provide a biodurable reticulatable elastomeric product. The product is designated as a polycarbonate polyurethane, being a polymer comprising urethane groups formed from, e.g., the hydroxyl groups of the polycarbonate polyol component and the isocyanate groups of the isocyanate component. In this embodiment, the process employs controlled chemistry to provide a reticulated elastomer product with good biodurability characteristics. The foam product employing chemistry that avoids biologically undesirable or nocuous constituents therein.

In one embodiment of the invention, the starting material of the porous biodurable reticulated elastomeric partially hydrophobic polymeric matrix contains at least one polyol component. For the purposes of this application, the term “polyol component” includes molecules comprising, on the average, about 2 hydroxyl groups per molecule, i.e., a difunctional polyol or a diol, as well as those molecules comprising, on the average, greater than about 2 hydroxyl groups per molecule, i.e., a polyol or a multi-functional polyol. Exemplary polyols can comprise, on the average, from about 2 to about 5 hydroxyl groups per molecule. In one embodiment, as one starting material, the process employs a difunctional polyol component. In this embodiment, because the hydroxyl group functionality of the diol is about 2. In another embodiment, the soft segment is composed of a polyol component that is generally of a relatively low molecular weight, typically from about 1,000 to about 6,000 Daltons. Thus, these polyols are generally liquids or low-melting-point solids. This soft segment polyol is terminated with hydroxyl groups, either primary or secondary.

Examples of suitable polyol components are polyether polyol, polyester polyol, polycarbonate polyol, hydrocarbon polyol, polysiloxane polyol, poly(ether-co-ester) polyol, poly(ether-co-carbonate) polyol, poly(ether-co-hydrocarbon) polyol, poly(ether-co-siloxane) polyol, poly(ester-co-carbonate) polyol, poly(ester-co-hydrocarbon) polyol, poly(ester-co-siloxane) polyol, poly(carbonate-co-hydrocarbon) polyol, poly(carbonate-co-siloxane) polyol, poly(hydrocarbon-co-siloxane) polyol, or mixtures of two or more thereof.

Useful polysiloxane polyols include oligomers of, e.g., alkyl and/or aryl substituted siloxanes such as dimethyl siloxane, diphenyl siloxane or methyl phenyl siloxane, comprising hydroxyl end-groups. Polysiloxane polyols with an average number of hydroxyl groups per molecule greater than 2, e.g., a polysiloxane triol, can be made by using, for example, methyl hydroxymethyl siloxane, in the preparation of the polysiloxane polyol component.

A particular type of polyol need not, of course, be limited to those formed from a single monomeric unit. For example, a polyether-type polyol can be formed from a mixture of ethylene oxide and propylene oxide. Additionally, in another embodiment, copolymers or copolyols can be formed from any of the above polyols by methods known to those in the art. Thus, the following binary component polyol copolymers can be used: poly(ether-co-ester) polyol, poly(ether-co-carbonate) polyol, poly(ether-co-hydrocarbon) polyol, poly(ether-co-siloxane) polyol, poly(ester-co-carbonate) polyol, poly(ester-co-hydrocarbon) polyol, poly(ester-co-siloxane) polyol, poly(carbonate-co-hydrocarbon) polyol, poly(carbonate-co-siloxane) polyol and poly(hydrocarbon-co-siloxane) polyol. For example, a poly(ether-co-ester) polyol can be formed from units of polyethers formed from ethylene oxide copolymerized with units of polyester comprising ethylene glycol adipate. In another embodiment, the copolymer is a poly(ether-co-carbonate) polyol, poly(ether-co-hydrocarbon) polyol, poly(ether-co-siloxane) polyol, poly(carbonate-co-hydrocarbon) polyol, poly(carbonate-co-siloxane) polyol, poly(hydrocarbon-co-siloxane) polyol or mixtures thereof. In another embodiment, the copolymer is a poly(carbonate-co-hydrocarbon) polyol, poly(carbonate-co-siloxane) polyol, poly(hydrocarbon-co-siloxane) polyol or mixtures thereof. In another embodiment, the copolymer is a poly(carbonate-co-hydrocarbon) polyol. For example, a poly(carbonate-co-hydrocarbon) polyol can be formed by polymerizing 1,6-hexanediol, 1,4-butanediol and a hydrocarbon-type polyol with carbonate.

Furthermore, in another embodiment of the invention, mixtures, admixtures and/or blends of polyols and copolyols can be used in the elastomeric matrix of the present invention. In another embodiment, the molecular weight of the polyol is varied. In another embodiment, the functionality of the polyol is varied.

In another embodiment of the invention, the starting material of the porous biodurable reticulated elastomeric partially hydrophobic polymeric matrix contains at least one isocyanate component and, optionally, at least one chain extender component to provide the so-called “hard segment”. For the purposes of this application, the term “isocyanate component” includes molecules comprising, on the average, about 2 isocyanate groups per molecule as well as those molecules comprising, on the average, greater than about 2 isocyanate groups per molecule. The isocyanate groups of the isocyanate component are reactive with reactive hydrogen groups of the other ingredients, e.g., with hydrogen bonded to oxygen in hydroxyl groups and with hydrogen bonded to nitrogen in amine groups of the polyol component, chain extender, crosslinker and/or water.

In another embodiment of the invention, the average number of isocyanate groups per molecule in the isocyanate component is about 2. In another embodiment of the invention, the average number of isocyanate groups per molecule in the isocyanate component is greater than about 2 is greater than 2.

The isocyanate index, a quantity well known to those in the art, is the mole ratio of the number of isocyanate groups in a formulation available for reaction to the number of groups in the formulation that are able to react with those isocyanate groups, e.g., the reactive groups of diol(s), polyol component(s), chain extender(s) and water, when present. In one embodiment of the invention, the isocyanate index is from about 0.9 to about 1.1. In another embodiment of the invention, the isocyanate index is from about 0.9 to about 1.02. In another embodiment, the isocyanate index is from about 0.98 to about 1.02. In another embodiment of the invention, the isocyanate index is from about 0.9 to about 1.0. In another embodiment of the invention, the isocyanate index is from about 0.9 to about 0.98.

The elastomeric polyurethane may contain from about 20 to 70% by weight of hard segment, preferably from about 25 to 35% by weight of hard segment and may contain from about 30 to 85% by weight of soft segment, preferably from about 50 to 80% by weight of soft segment, based upon the total weight of the polyurethane.

Exemplary diisocyanates include aliphatic diisocyanates, isocyanates comprising aromatic groups, the so-called “aromatic diisocyanates”, and mixtures thereof. Useful aliphatic diisocyanates include tetramethylene diisocyanate, cyclohexane-1,2-diisocyanate, cyclohexane-1,4-diisocyanate, hexamethylene diisocyanate, isophorone diisocyanate, methylene-bis-(p-cyclohexyl isocyanate) (“H₁₂ MDI”), and mixtures thereof. Useful aromatic diisocyanates include p-phenylene diisocyanate, 4,4′-diphenylmethane diisocyanate (“4,4′-MDI”), 2,4′-diphenylmethane diisocyanate (“2,4′-MDI”), 2,4-toluene diisocyanate (“2,4-TDI”), 2,6-toluene diisocyanate(“2,6-TDI”), m-tetramethylxylene diisocyanate, and mixtures thereof.

In one embodiment of the invention, the isocyanate component contains a mixture of at least from about 5% to 50% by weight of 2,4′-MDI and with from about 50 to 95% by weight of 4,4′-MDI, based upon the total weight of the component. Without being bound by any particular theory, it is thought that the use of higher amounts of 2,4′-MDI in a blend with 4,4′-MDI results in a softer elastomeric matrix because of the disruption of the crystallinity of the hard segment arising out of the asymmetric 2,4′-MDI structure.

In another embodiment of the invention, the starting material of the porous biodurable reticulated elastomeric partially hydrophobic polymeric matrix contains suitable chain extenders, preferably for the hard segments, including, but not limited to, diols, diamines, alkanol amines and mixtures thereof In another embodiment of the invention, the chain extender is an aliphatic diol having from 2 to 10 carbon atoms. In another embodiment of the invention, the diol chain extender is selected from the group consisting of ethylene glycol, 1,2-propane diol, 1,3-propane diol, 1,4-butane diol, 1,5-pentane diol, diethylene glycol, triethylene glycol, and mixtures thereof. In another embodiment of the invention, the chain extender is a diamine having from 2 to 10 carbon atoms. In another embodiment of the invention, the diamine chain extender is selected from the group consisting of ethylene diamine, 1,3-diaminobutane, 1,4-diaminobutane, 1,5 diaminopentane, 1,6-diaminohexane, 1,7-diaminoheptane, 1,8-diaminooctane, isophorone diamine and mixtures thereof. In another embodiment of the invention, the chain extender is an alkanol amine having from 2 to 10 carbon atoms. In another embodiment of the invention, the alkanol amine chain extender is selected from the group consisting of diethanolamine, triethanolamine, isopropanolamine, dimethylethanolamine, methyldiethanolamine, diethylethanolamine, and mixtures thereof.

In one embodiment of the invention, the starting material of the porous biodurable reticulated elastomeric partially hydrophobic polymeric matrix contains a small quantity of an optional ingredient, such as a multi-functional hydroxyl compound or other crosslinker having a functionality greater than 2, e.g., glycerol, is present to facilitate crosslinking. In another embodiment of the invention, the optional multi-functional crosslinker is present in an amount just sufficient to achieve a stable foam, i.e., a foam that does not collapse to become non-foamlike. Alternatively, or in addition, polyfunctional adducts of aliphatic and cycloaliphatic isocyanates can be used to impart crosslinking in combination with aromatic diisocyanates. Alternatively, or in addition, polyfunctional adducts of aliphatic and cycloaliphatic isocyanates can be used to impart crosslinking in combination with aliphatic diisocyanates.

In one embodiment of the invention, the starting material of the porous biodurable reticulated elastomeric partially hydrophobic polymeric matrix is a commercial polyurethane polymer. Polyurethane polymers are linear, not crosslinked, polymers, and therefore they are soluble, can be melted, readily analyzable, and readily characterizable. In this embodiment of the invention, the staring polymer provides good biodurability characteristics. The reticulated elastomeric matrix is produced by taking a solution of the commercial polymer such as polyurethane and charging it into a mold that has been fabricated with surfaces defining a microstructural configuration for the final implant or scaffold, solidifying the polymeric material and removing the sacrificial mold by melting, dissolving or subliming-away the sacrificial mold. The foam product employing a foaming process that avoids biologically undesirable or nocuous constituents therein.

Of particular interest are thermoplastic elastomers such as polyurethanes whose chemistry is associated with good biodurability properties, for example. In one embodiment of the invention, such thermoplastic polyurethane elastomers include polycarbonate polyurethanes, polyester polyurethanes, polyether polyurethanes, polysiloxane polyurethanes, polyurethanes with so-called “mixed” soft segments, and mixtures thereof. Mixed soft segment polyurethanes are known to those skilled in the art and include, e.g., polycarbonate-polyester polyurethanes, polycarbonate-polyether polyurethanes, polycarbonate-polysiloxane polyurethanes, polyester-polyether polyurethanes, polyester-polysiloxane polyurethanes and polyether-polysiloxane polyurethanes. In another embodiment of the invention, the thermoplastic polyurethane elastomer comprises at least one diisocyanate in the isocyanate component, at least one chain extender and at least one diol, and may be formed from any combination of the diisocyanates, difunctional chain extenders and diols described in detail above.

In one embodiment of the invention, the weight average molecular weight of the thermoplastic elastomer is from about 30,000 to about 500,000 Daltons. In another embodiment of the invention, the weight average molecular weight of the thermoplastic elastomer is from about 50,000 to about 250,000 Daltons.

Some suitable thermoplastic polyurethanes for practicing the invention, in one embodiment suitably characterized as described herein, include, but are not limited to, polyurethanes with mixed soft segments comprising polysiloxane together with a polyether and/or a polycarbonate component, as disclosed by Meijs et al. in U.S. Pat. No. 6,313,254; and those polyurethanes disclosed by DiDomenico et al. in U.S. Pat. Nos. 6,149,678, 6,111,052 and 5,986,034, all of which are incorporated herein by reference.

Some commercially-available thermoplastic elastomers suitable for use in practicing the present invention include the line of polycarbonate polyurethanes supplied under the trademark BIONATE® by The Polymer Technology Group Inc. (Berkeley, Calif.). For example, the very well-characterized grades of polycarbonate polyurethane polymer BIONATE® 80A, 55 and 90 are soluble in THF, processable, reportedly have good mechanical properties, lack cytotoxicity, lack mutagenicity, lack carcinogenicity and are non-hemolytic. Another commercially-available elastomer suitable for use in practicing the present invention is the CHRONOFLEX® C line of biodurable medical grade polycarbonate aromatic polyurethane thermoplastic elastomers available from CardioTech International, Inc. (Woburn, Mass.). Yet another commercially-available elastomer suitable for use in practicing the present invention is the PELLETHANE® line of thermoplastic polyurethane elastomers, in particular the 2363 series products and more particularly those products designated 81A and 85A, supplied by The Dow Chemical Company (Midland, Mich.). These commercial polyurethane polymers are linear, not crosslinked, polymers, therefore, they are soluble, readily analyzable and readily characterizable.

Coatings and Delayed Drug Delivery

A foam composite according to the invention can comprise a scaffold of reticulated, open cell hydrophobic and preferably biostable material having a plurality of surfaces defining a plurality of pores, and a coating of a substantially hydrophilic foam material disposed upon the surfaces of the hydrophobic foam and within pores. In another embodiment, a foam composite according to the invention can comprise a scaffold of reticulated, open cell hydrophobic material having a plurality of surfaces defining a plurality of pores, and a coating of a substantially hydrophilic material layer in the form or a film or coating disposed upon the surfaces of the hydrophobic foam and within pores. The hydrophilic foam or the hydrophilic material film or coating can be polymeric in nature. The polymer forming the film or the coating can be both non-biodegradable and degradable. The reticulated nature of the scaffold is advantageous due to the characteristic large surface area, which is suitable for carrying a coating and/or large quantities of therapeutic agents. The cells or pores in the hydrophobic foams may vary in their degree of openness or interconnection (reticulation) depending upon the application. Open cell hydrophobic foams may have a reticulated, substantially reticulated, or a non-reticulated structure. Hydrophobic foams having a more open, reticulated structure lend themselves to applications in which a gas or liquid is passed through the structure, as in a filter, and where fluid flow and pressure drop considerations are of particular importance. Such foam composite exhibits structural characteristics of the hydrophobic foam and absorbency characteristics of the hydrophilic foam or a hydrophilic layer. Those skilled in the art will understand how to vary the degree of openness as well as the pore size of pores.

To facilitate immobilization of the drug on the scaffold, the scaffold may be hydrophilized or coated with a hydrophilic coating to facilitate attachment of therapeutic agent or therapeutic agent drug bearing structures such as biologically erodible microspheres, microcapsules or other micropackages. Hydrophilization may comprise treatment of the hydrophobic material to render the surfaces partially hydrophilic or application of an adhesive or application of a hydrophilic coating, or deposit of a hydrophilic foam, for example, as described in Thomson, U.S. Pat. No. 6,617,014, incorporated herein by reference. In another embodiment, hydrophilization may comprise a combination of treatment of the hydrophobic material to render the surfaces partially hydrophilic, application of an adhesive, application of a hydrophilic coating, and deposit of a hydrophilic foam.

The hydrophilic foam coating can be made from polyuretanes containing appropriate and suitable isocyanate and polyols. Isocyanates suitable for this invention are aromatic, such as, for example, toluene dilsocyanate (TDI) or methylene diphenyl isocyanate (MDI), or with a aliphatic duisosyanate, such as hydrogenated MDI or isopherone dilsocyanate. One example of polyol is polyether polyols which are homopolymers of ethylene oxide, also known as polyethylene glycols, or copolymers of ethylene oxide and propylene oxides. Other examples of suitable polyols are polyester polyol, poly(ether-co-ester) polyol, poly(ether-co-hydrocarbon) polyol, poly(ether-co-siloxane) polyol, poly(ester-co-siloxane) polyol,poly(ether-co-carbonate) polyol, poly(ester-co-carbonate) polyol, poly(ester-co-hydrocarbon) polyol, or mixtures thereof.

Hydrophilic polyurethanes foams are preferably made by the so-called pre-polymer or pseudo pre-polymer method. In this technique, the polyol and the isocyanate are reacted in various ratios and by various reaction schemes to produce an intermediate product called a pre-polymer or quasi pre-polymer. This is then emulsified in an aqueous phase to produce the final foam coating. In another embodiment of the invention, the hydrophilic foam coating is prepared by contacting with a solution of a prepolymer in a solvents, such as DMF,or DMAC or NMP, by coating, spraying or dipping and contacting, and then the coated or otherwise prepolymer impregnated reticulated hydrophobic polyurethane is squeezed or spread or dispersed and optionally hung in place to remove the excess prepolymer solution followed by air drying or placing under vacuum to remove the solvent and finally curing in contact with water. The curing can be accomplished a water bath or in a humidity chamber or any space with sufficient environmental humidity.

Prepolymers suitable for use in the present invention are isocyanate-capped polyether prepolymers with an NCO functionality of greater than 5% as more particularly described below. The prepolymers are preferably based on polyether polyols capped with aromatic isocyanates such as for example toluene diisocyanate (TDI) or methylene diphenyl isocyanate (MDI) or with aliphatic isocyanates, such as, for example isopherone diisocyanate (IPDI) or hydrogenated methylene diphenyl isocyanate (HMDI). The polyether polyols are hydrophilic polyoxyalkylenes with a minimum of 40 mole % ethylene oxide. Isocyanate-capped polyether prepolymers which have been found to be suitable for use in the practice of the present invention include without limitation prepolymers commercially available or can be manufactured. Other suitable polyols are polyester polyol, poly(ether-co-ester) polyol, poly(ether-co-hydrocarbon) polyol, poly(ether-co-siloxane) polyol, poly(ester-co-siloxane) polyol,poly(ether-co-carbonate) polyol, poly(ester-co-carbonate) polyol, poly(ester-co-hydrocarbon) polyol, polycarbonate polyol, hydrocarbon polyol, polysiloxane polyol, poly(carbonate-co-hydrocarbon) polyol, poly(carbonate-co-siloxane) polyol, poly(hydrocarbon-co-siloxane) polyol, or mixtures thereof.

Hydrophilic polyurethane coatings can also be prepared from solvent systems as well as water. For solvent borne coatings, the linear polyurethane is first dissolved in the appropriate solvent, such as tetrahydrofuran, N-methylpyrolidone, dimethyl formamide, dimethylacetamide, etc. at concentrations from about 1 to 40 wt % solids and preferably in the 1 to 10 wt % solid. Coatings are then simply cast on a suitable substrate and heated (atmospheric pressure or a vacuum) to evaporate the solvent, leaving a coating of the polyurethane. Alternatively as described before, solvent borne coatings may be prepared by the prepolymer method by dissolving a urethane prepolymer in the suitable solvents such as tetrahydrofuran, N-methylpyrolidone, dimethyl formamide, dimethylacetamide as well as a number of aromatic solvents, such as toluene, xylene, etc.). Chain extenders and/or crosslinkers and catalyst are then added, stirred in and coatings cast. They are then heated to both evaporate the solvent as well as chain extend/crosslink (cure) the polyurethane. Higher concentrations may be used, up to over 50% by weight of solids. Coatings may also be formed in a similar fashion bye first dissolving the polyol, chain extender, crosslinker and catalyst in solvent and then adding the isocyanate, followed by casting and curing. High concentrations are also possible with this method.

Polyurethane coatings may also be prepared from water-based systems (dispersions). Polyurethanes used are ionomers (cationic or anionic) or, less often, from poly urethanes containing hydrophilic chains. Cationic ionomers are synthesized by the reaction of isocyanate-terminated prepolymers with tertiary amines containing hydroxyl groups, followed by quaternization of the tertiary nitrogen atom with, for example, methyl sulphate, alkyl chlorides, benzyl chloride, etc. This is then dispersed in water. Anionic ionomers are synthesized by the reaction of isocyanate-terminated prepolymers with salts of carboxylic or sulfonic acids which incorporate two reactive groups, amine or hydroxyl. The acid groups are first converted into salts to prevent their reaction with isocyanate. The resulting ionomer is also dispersible in water. Alternatively, if anionic ionomers are prepared using carboxylic acids with amine groups, the reaction may be carried out in water (the amine groups will react with the isocyanate groups much faster than does water). Typical concentrations are in the range of 30-60% solids. In one embodiment the hydrophilic film or coating for the internal surfaces of the hydrophobic elastomeric material that is used to fabricate the hydrophobic scaffold or the implant of this invention can be made from flowable polymeric material such as a polymer solution, emulsion, microemulsion, suspension, dispersion, a liquid polymer, or a polymer melt. For example, the flowable polymeric material can comprise a solution of the polymer in a volatile organic solvent. The coating or the film can have additional capacity to transport or bond to active ingredients that can then be preferentially delivered.

In one embodiment, the polymeric material can comprise a thermoplastic elastomer and the flowable polymeric material can comprise a solution of that thermoplastic elastomer that can also be biodurable. In another embodiment, the polymeric material can comprise a solvent-soluble biodurable thermoplastic elastomer and the flowable polymeric material can comprise a solution of that solvent-soluble biodurable thermoplastic elastomer. The solvent can then be removed or allowed to evaporate to solidify the polymeric material into a film or coating. Solidifying the polymeric material into a film or a coating can be optionally assisted by vacuum and/or heating to a temperature below the softening temperatures of the polymer or of the substrate material. If sufficiently volatile, the solvent may be allowed to evaporate off, e.g., overnight.

In one embodiment, solvents are biocompatible and sufficiently volatile to be readily removed. The solvent or solvent blend for the coating solution is chosen with consideration given to, inter alia, the proper balancing the viscosity, deposition level of the polymer, wetting rate and evaporation rate of the solvent to properly coat on elastomeric matrix that is used to fabricate the implant of this invention, as known to those in the art. In one embodiment, the solvent is chosen such the polymer is soluble in the solvent. In another embodiment, the solvent is substantially completely removed from the coating. In another embodiment, the solvent is non-toxic, non-carcinogenic and environmentally benign. Mixed solvent systems can be advantageous for controlling the viscosity and evaporation rates. In all cases, the solvent should not preferably react with the coating polymerSuitable solvents, depending, of course, upon the solubility of the polymer, include THF, DMF, DMAC, DMSO, dioxane and N-methyl-2-pyrrolidone or their mixtures thereof. Additional suitable solvents will be known to those skilled in the art.

Furthermore, one or more coatings may be applied by contacting with a film-forming biocompatible polymer either in a liquid coating solution or in a melt state under conditions suitable to allow the formation of a biocompatible polymer film. In one embodiment, the polymers used for such coatings are film-forming biocompatible polymers with sufficiently high molecular weight so as to not be waxy or tacky. The polymers should also adhere substantially to the hydrophilic solid phase of the reticulated elastomeric matrix that is used to fabricate the implant. In another embodiment, the bonding strength is such that the polymer film does not crack or dislodge during handling or deployment of the implant.

The coating on the outer surface can be made from a biocompatible polymer, which can include be both biodegradable and non-biodegradable polymers. The coating on elastomeric matrix that is used to fabricate the implant of this invention can be applied by, e.g., dipping or spraying a coating solution comprising a polymer or a polymer that is admixed with a pharmaceutically-active agent. In one embodiment, the polymer content in the coating solution is from about 1% to about 40% by weight. In another embodiment, the polymer content in the coating solution is from about 1% to about 20% by weight. In another embodiment, the polymer content in the coating solution is from about 1% to about 10% by weight. In another embodiment, the layer(s) and/or portions of the outermost surface not being solution-coated are protected from exposure by covering them during the solution-coating of the outermost surface.

Suitable film-forming biodurable biocompatible non-biodegradable polymers to be used for hydrophilic coating include polyamides, polyolefins (e.g., polypropylene, polyethylene), nonabsorbable polyesters (e.g., polyethylene terephthalate), silicones, poly(meth)acrylates, polyesters, polyalkyl oxides (e.g., polyethylene oxide), polyvinyl alcohols, polyethylene glycols and polyvinyl pyrrolidone, as well as hydrogels, such as those formed from crosslinked polyvinyl pyrrolidinone and polyesters. Other polymers, of course, can also be used as the biocompatible polymer provided that they can be dissolved, cured or polymerized. Such polymers and copolymers include polyolefins, polyisobutylene and ethylene-α-olefin copolymers; acrylic polymers (including methacrylates) and copolymers; vinyl halide polymers and copolymers, such as polyvinyl chloride; polyvinyl ethers, such as polyvinyl methyl ether; polyvinylidene halides such as polyvinylidene fluoride and polyvinylidene chloride; polyacrylonitrile; polyvinyl ketones; polyvinyl aromatics such as polystyrene; polyvinyl esters such as polyvinyl acetate; copolymers of vinyl monomers with each other and with α-olefins, such as etheylene-methyl methacrylate copolymers and ethylene-vinyl acetate copolymers; acrylonitrile-styrene copolymers; ABS resins; polyamides, such as nylon 66 and polycaprolactam; alkyd resins; polycarbonates; polyoxymethylenes; polyimides; polyethers; epoxy resins; polyurethanes; rayon; rayon-triacetate; cellophane; cellulose and its derivatives such as cellulose acetate, cellulose acetate butyrate, cellulose nitrate, cellulose propionate and cellulose ethers (e.g., carboxymethyl cellulose and hydoxyalkyl celluloses); and mixtures thereof.

Suitable film-forming biodurable biocompatible biodegradable polymers to be used for hydrophilic coating include bioabsorbable aliphatic polyesters (e.g., homopolymers and copolymers of lactic acid, glycolic acid, lactide, glycolide, para-dioxanone, trimethylene carbonate, ε-caprolactone and blends thereof). Further, biocompatible polymers include film-forming bioabsorbable polymers; these include aliphatic polyesters, poly(amino acids), copoly(ether-esters), polyalkylenes oxalates, polyamides, poly(iminocarbonates), polyorthoesters, polyoxaesters including polyoxaesters containing amido groups, polyamidoesters, polyanhydrides, polyphosphazenes, biomolecules and blends thereof. For the purpose of this invention aliphatic polyesters include polymers and copolymers of lactide (which includes lactic acid d-, l- and meso lactide), ε-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one and blends thereof.

Hydrophilic coatings or layers made from polymers such as partially hydrophilic polyurethane is compatible with and absorbs water while conventional resiliently compressible polyurethanes are hydrophobic and shed water. While their hydrophilic nature gives hydrophilic coatings such as hydrophilic partially polyurethane useful properties such as the ability to absorb aqueous liquids, and serve as a carrier and aqueous flow medium for biologically active agents, it also leads to certain deficiencies. Among these are low physical strength, poor cell size control, and relatively high densities. Furthermore, the hydrophilic layer in forms such as foam or films swells considerably upon absorption. Hydrophobic polyurethane is compressible but does not hold liquid. With regard to the preferred composite foam material described above, the hydrophilic coating provides for the composite foam with a hydrophilic character, while the reticulated hydrophobic foam substrate can provide the composite foam with physical strength and sufficiently good flow-through characteristics that characterize a reticulated foam. Thus, while a hydrophilic coating may swell when it absorbs water or another liquid, the reticulated hydrophobic scaffold can be sufficiently strong to maintain integrity and prevent any significant increase in the overall size of the composite articles due to swelling, even when exposed to aqueous fluids for extended periods. Also, the absence of swelling enhances removal by compressing, collapsing, gripping and withdrawal.

It is understood that this method may be applied to any type of hydrophilic material such as hydrophilic polyurethane which is supported by a fluid permeable biodurable structural support such, for example, as foam, woven or nonwoven fabric or networked composites of microstructural elements of various forms such as rods, tubes, tubules, fusiforms, helices, cylinders, footballs, bullets, and so on.

The film-forming polymer coating or the foamed coating to coat reticulated elastomeric biodurable matrix or the scaffold of the implant of this invention can provide a vehicle for the delivery of and/or the controlled release of a pharmaceutically-active agent, for example, a drug or a microspheres containing drug. In another embodiment, the pharmaceutically-active agent is admixed with, covalently bonded to and/or adsorbed in or on the coating of reticulated elastomeric biodurable matrix to provide a pharmaceutical composition or by incorporating the pharmaceutically-active agent into additional hydrophilic coatings.

In one embodiment, the coating polymer or the coating foam and pharmaceutically-active agent or microspheres containing pharmaceutically-active agent have a common solvent. This can provide a coating that is a solution. In another embodiment, the pharmaceutically-active agent can be present as a solid dispersion in a solution of the coating polymer in a solvent. Alternatively, a pharmaceutically-active agent can be coated onto the foam, in one embodiment, using a pharmaceutically-acceptable carrier. If melt-coating is employed, then, in another embodiment, the pharmaceutically-active agent withstands melt processing temperatures without substantial diminution of its efficacy.

In another embodiment, a top coating can be applied to delay release of the pharmaceutically-active agent or microspheres containing pharmaceutically-active agent. In another embodiment, a top coating can be used as the matrix for the delivery of a second pharmaceutically-active agent. A layered coating, comprising respective layers of fast- and slow-hydrolyzing polymer, can be used to stage release of the pharmaceutically-active agent or to control release of different pharmaceutically-active agents placed in the different layers. Polymer blends may also be used to control the release rate of different pharmaceutically-active agents or to provide a desirable balance of coating characteristics (e.g., elasticity, toughness) and drug delivery characteristics (e.g., release profile). Polymers with differing solvent solubilities can be used to build-up different polymer layers that may be used to deliver different pharmaceutically-active agents or to control the release profile of a pharmaceutically-active agents.

A reticulated elastomeric biodurable matrix or the scaffold of the implant of this invention comprising a pharmaceutically-active agent may be formulated by mixing one or more pharmaceutically-active agent with the polymer used to make the scaffold, with the solvent or with the polymer-solvent mixture and foamed. In another embodiment, the components, polymers and/or blends used to form the foam comprise a pharmaceutically-active agent. To form these foams, the previously described components, polymers and/or blends are admixed with the pharmaceutically-active agent prior to forming the foam or the pharmaceutically-active agent is loaded into the foam after it is formed.

A preferred drug delivery implant material for use in the present invention is a resiliently compressible composite polyurethane foam comprising a hydrophilic polymer foam coated on and throughout the pore surfaces of a nonabsorbable hydrophobic foam scaffold. One suitable such material is a composite polyurethane foam product as disclosed and claimed in Thomson, U.S. Pat. No. 6,617,014, which is both compressible and water absorbent or liquid absorbent. The hydrophobic foam provides tensile strength, support and resilient compressibility enabling the desired collapsing of the drug delivery implant for delivery and reconstitution in situ. The hydrophilic foam coated on the interior pore surfaces of the hydrophobic foam can support useful quantities of a drug for release in situ. A particular material of this nature is identified by the trademark CO-FOAMJ (Hydrophilix, LLC, Portland, Me. (USA)) and is referenced herein as the “CO-FOAMJ composite” or the “CO-FOAMJ foam composite”.

Useful flexible at least patrially hydrophobic polyurethane foams and hydrophilic polymeric coatings would be known to those skilled in the art. Representative and preferred embodiments of such porous drug-bearing materials and composites suitable for use as implant materials are set forth in co-pending, commonly assigned U.S. provisional patent application Ser. No. 60/471,518, filed May 15, 2003, and U.S. provisional patent application Ser. No. 60/471,520, filed May 15, 2003, both of which are incorporated herein by reference in their entirety, especially with regard to the disclosure and teaching of such composite foams and coatings.

Preferred composite foams have a composition that allows relatively free flow of urine through the foam implant. Additionally the resiliency of the foam composite helps retain the drug delivery implant in place as bladder 10 naturally contracts and expands.

Desired drugs may be incorporated into an implant in any suitable manner. In a preferred embodiment an implant such as implant 42 or another suitable shape such as a cylinder, sphere, bullet, football, or irregular shape, comprises a porous or apertured structural scaffold coated with therapeutic agent-bearing material that releases one or more therapeutic agents.

The therapeutic agent or agents, or therapeutic agent-bearing structures, may be adhered, incorporated in a hydrophilic foam or other coating on the hydrophobic scaffold, or, possibly covalently bonded to the hydrophobic scaffold or the coating.

More specifically, embodiments of the invention enable the delivery of therapeutic and other biologically useful molecules from micro drug delivery systems such as microspheres, microcapsules, microspherules and other such micropackages, liposomes, nanoparticles, biodegradable controlled release polymer matrices, and other such drug or biologic agent micropackaging systems, as are known, or may become known, to those skilled in the art which are collectively referenced herein as “microspheres.” Preferred microspheres for use in the invention can be charged with a biologically useful agent and will biodegrade or bioerode to release the agent in a controlled manner.

The agents to be delivered may include one or more small molecules, macromolecules, liposomal encapsulations of molecules, microdrug delivery system encapsulation of therapeutic molecules, covalent linking of carbohydrates and other molecules to therapeutic molecules, and gene therapy preparations. The microspheres or microcapsules may contain therapeutic agents, enzymes, or other compounds for the purpose of delayed, sustained, or otherwise controlled release.

There are several general types of controlled release systems that can be employed. For example, therapeutic agent release can be diffusion controlled, meaning that the diffusion of the agent trapped within a polymer matrix is the rate-determining factor for the overall release rate. Erosion based systems also exist in which a polymer degrades over time and releases a therapeutic agent in an amount proportional to the gradual erosion. An osmotic pumping device uses osmotic pressure as the driving force for release. A fourth system is based on the swelling of a polymeric matrix, such as a hydrogel. Hydrogels are polymers that absorb and swell in an aqueous environment. The release of the agent is dependent on the volume increase of the gel upon swelling and is then diffusion controlled.

In a preferred embodiment, microspheres are embedded within a layer of hydrophilic polyurethane matrix or a layer or other hydrophilic degradable and non-degradable polymer matrix or layer applied to the surface of a reticulated polyurethane scaffold or other stable support. It is contemplated that the embedding of microspheres may be within any hydrophilic polyurethane or other hydrophilic degradable and non-degradable polymer, whether it is alone or applied to any stable surface.

In one embodiment of preparing the microsphere-bearing composite foam material of the invention, microspheres can be mixed with the free polymer components of the hydrophilic polyurethane, in the prepolymer phase. In another embodiment embodiment of preparing the microsphere-bearing composite foam material of the invention, microspheres can be mixed with the film or coating forming hydrophilic polymer during the solution preparing process. In another embodiment, polyurethane, solvent, and a therapeutic agent are added as a coating, and then the solvent is evaporated, leaving behind a coating with embedded microspheres. The resultant mixture can then be used to coat hydrophobic scaffold, fixedly embedding microspheres within a hydrophilic layer, as it cures. By mixing microspheres within the hydrophilic layer, a dispersion of microspheres throughout the hydrophilic layer coated on the surfaces of pores of the hydrophobic support can be obtained. Beneficially, microspheres are substantially held in place within hydrophilic polyurethane surface layer through covalent or other chemical bonding, or mechanical restraint

Substantial amounts of therapeutic agent may be incorporated within hydrophilic layer as compared to merely covalently binding agent directly to carrier. Furthermore, the inclusion of microspheres in polyurethane coating exposes microspheres to whatever solution carrier was immersed within or exposed to. With both an aqueous solution or a lipid solution, microspheres are exposed to hydrated hydrophilic polyurethane layer of carrier and eluted into a liquid environment thereby allowing microspheres to be degraded and release agent in a controlled fashion from the hydrophilic polyurethane. This is in direct contrast to covalently binding or adsorbing these drugs to the hydrophilic layer, which may result in unexpected or uncontrollable release of therapeutic agent. The reticulated array of struts of carrier allows quick and easy fluidic transmission of therapeutic agent. Such therapeutic agents may include, but are not limited to, pharmaceuticals, therapeutic substances, vaccines, prophylactics and other substances depending on the intended use or result.

Immobilization of microspheres in hydrophilic layer of carrier is thus achieved without adhesive. Hydrophilic layer acts as a binder and when the layer becomes fully hydrated, it remains attached to underlying scaffold does not impede the release of drugs or compounds from microspheres as they degrade, or utilize another mechanism to release an agent over time, based on their own internal characteristics.

The composition of hydrophilic layer is selected for its permeability to the particular agent being dispersed by the invention. Such materials are well-known. Such materials are generally of a molecular structure which includes interstices, i.e., pores or voids, large enough to quickly allow absorption and relatively free movement of water molecules through the hydrophilic materials. In addition, in accordance with the invention, the material, of which hydrophilic coating is made, should have interstices large enough to allow transmission of agent being dispersed, typically as a solution in an aqueous medium that has permeated contents of the bladder in the coating, for example, the case of a medication dispersing from device 10 situated in the human bladder.

Delivering agent locally generally results in a very small amount of agent being required to treat a specific location within the tissue, which has substantial benefits, such as less side effects. Smaller doses of agent will minimize the need to replace the device as often and will reduce the systemic effects that result from large drug doses as well as the effects that the agents will have on normally functioning tissue.

When the hydrophilic coating is in an aqueous medium, liquid is permeated throughout hydrophilic coating and its surrounding microspheres, and microspheres are working in an aqueous medium. Hydrophilic layer largely has the characteristics of a hydrogel. Thus in the case of microspheres which release agent in response to degradation of their cores, water in coating causes the characteristic hydrolytic activity of the aqueous phase, which degrades microspheres and releases therapeutic agent in a controlled fashion.

The system can potentially allow the storage and immobilization of a large quantity of therapeutic agents within the hydrophilic layer of polyurethane, possibly a greater quantity than could be readily loaded into a similar volume or weight of hydrophilic polyurethane alone, without a hydrophobic polyurethane scaffold, simply by adsorption or covalently bonding of the agent to the material. Furthermore, the encapsulation of the microspheres by hydrophilic polyurethane may not substantially change the microsphere release properties because hydrophilic layer can be expected to become fully hydrated and the equivalent of a hydrogel for that purpose to allow for fluid transport without losing its integrity.

When a coating or therapeutic agent carrying matrix is hydrophilic, it will absorb water and it will eventually degrade biogradable components of microspheres and release therapeutic agents at a controlled rate. Large amounts of these microspheres may be stored in the hydrophilic layer of carrier with microspheres that are programmed to release therapeutic agent in a controlled fashion. Depending on the intended use, hydrophilic layer may be filled to varying degrees, from very few to fully packed with microspheres for the purpose of delivery of therapeutic agents.

The preferred composite foam carrier uses hydrophobic layer as a physical, three-dimensional, reticulated, flow-through scaffold for support and for storage of additional microspheres or other material in pores. Preferably, there is little or no reaction of the agent in microsphere until it is released to perform its function. Accordingly, the preferred composite foam scaffolding is an inert support structure. The hydrophobic layer may be enhanced by addition of other materials, including polymers, which enhance its desirable properties.

An advantage of using preferred composite foam or another reticulated foam for a scaffold is that because of its open flow-through characteristic, the compounds are released from microspheres over the entire internal and external surface area of the preferred composite foam and are available to be dispersed within any solution that passes through the material. This is in contrast to a conventional hydrophilic polyurethane, which has relatively poor flow-through characteristic and relatively poor mechanical integrity. As a result, in that setting, microspheres embedded within the center of the hydrophilic polyurethane, which release their therapeutic agent, requires diffusion of that therapeutic agent through the entire mass of the hydrophilic polyurethane to reach the surface and then be dispersed within the solvent. This is because hydrophilic polyurethane does not have a reticulated open-cell structure. Thus a larger amount of therapeutic agent can be delivered through device 10 over a longer period of time as compared to alternative structures.

Since microspheres degrade and release a therapeutic agent or agents (for example, drops or other water soluble agents) into hydrophilic layer from which they exit the carrier, the concentration is most intense at the surface of preferred composite foam. This may be particularly useful with respect to surface applications of therapeutic agents for the purposes of wound healing or intravaginal therapeutic agent delivery, or other mucosal therapeutic agent delivery.

Microspheres allow a highly concentrated solution of agent to be dispersed in comparison with systems where the same therapeutic agent or the same chemical is either absorbed or embedded or by desiccation concentrated it in the hydrophilic layer. Furthermore, in absorption or absorption or embedding of a compound in a hydrophilic layer, the release kinetics are dramatically different then from the release kinetics of microspheres. Without microspheres, the release kinetics generally comprise a first order release, a dramatic drop-off, and then an additional drop off to zero over a period of time. By using microspheres, agent release can be more accurately controlled by using microspheres with different release characteristics.

Microsphere release of a therapeutic agent has certain advantages as compared to release directly by a foam. For example, such release avoids the uncertainties created by degradation of composite foam or the degradation of an adhesive over a period of time, pH variation at the delivery site, or movement of the foam.

A device of the invention is useful for a number of applications. Specifically, a device may be inserted into a bodily cavity and placed next to or even shaped around various types of indwelling devices, such as heart valves, pacemakers, artificial joints, intravenous or intraarterial catheters or devices that are inserted into the body cavity such as gastrointestinal tubes, intrauterine devices, or diaphragms. Microspheres can also be triggered to release biologically active agents when the pH of the environment turns either acidic or basic. This change in pH may be due to changes occurring naturally in the environment or changes artificially induced. Urinary catheters, including Foley catheters and catheters that have no balloon, and ureteral stents, may be used according to this disclosure to prevent urease activity or prevent bacterial infestation. The device would be placed in an environment where aqueous solution (such as blood) or lipids pass through pores.

In an alternative embodiment, the inventive device comprises a controlled release formulation comprising microspheres of a vaccine suspended in a hydrophilic polymer matrix for delivering appropriate antigens for immunization against an infectious disease. In traditional methods, the efficiency of such vaccines often is low because of rapid degradation of antigens and their very short in-vivo half lives. Thus, large doses have been required to achieve adequate local concentrations. An advantage of the microencapsulation is that it protects the potency of weak antigens such as the small synthetic or recombinant peptides of HIV. Another advantage is that it may, by virtue of the improved delivery of antigen to the immunologic system, enhance the speed, rigor and persistence of the immune response. A further advantage may be modulation of antibody avidity, specificity, quantity, isotype and subclass. Furthermore the amount of antigen needed to provide effective protection may be decreased, thereby decreasing the cost of the vaccines. Additionally, the microspherical delivery form of the vaccine pursuant to the invention, may be more efficacious than a conventional aqueous vaccine.

In general, the quantity of therapeutic agent and the micropackaging, if employed, are selected according to the anticipated rate of elution from an implant according to the invention to provide a desired dosage throughout the intended implant period. The therapeutic agent-supporting capacity of an implant may be varied by varying its mass within a given external periphery, for example, by varying the thickness of an implant such as implant 42 as determined by the spacing between the inner and outer surfaces 52 and 54, or by changing the shape of inner surface 52 or by increasing the temperature or enlarging the size.

If desired, measures may be taken to modify the gross density of implant 42 to render it buoyant in urine, so that it will tend to migrate upwardly in bladder 10, away from trigone 22, when the host is upright. For example the material or materials employed to fabricate an implant such as implant 42 may be selected to provide an implant of a desired gross density. The term “gross density” is used to refer to the overall density of the implanted product, referring to its displacement in water.

Alternatively density control materials, such for example, EXPANCEL7 gas filled microspheres, available from the Casco Products unit Akzo Nobel, may be included in the structure of an implant.

The implant according to the invention may be of any suitable size and will normally be sized according to the target implantation site. For example, an intravesicular implant may have a major and/or minor diameter in the range of from about 0.5 to about 12 cm, preferably about 3 to about 8 cm, and more preferably about 4 to about 6 cm. Height 46, as a proportion of a diameter may lie in the range of from about 0.1 to about 1.0, preferably about 0.2 to about 0.6, more preferably about 0.4 to about 0.5 cm.

In a preferred embodiment of the invention the biologically active substance is covalently bound to the hydrophilic material. The degradable hydrophilic material will be absorbed nearby, causing it to degrade by hydrolysis in a predictable fashion. This hydrolysis reaction may create a relatively acidic environment within bladder 10 which can be useful in reducing calcification, the formation of stones and the like.

Therapeutic Agents and Therapies

The invention also provides therapeutic agent delivery implants loaded with complex therapeutic agent formulations which may comprise, for example, one or more active therapeutic agents together with one or more adjuvants to facilitate the performance of at least one of the therapeutic agents. For example, an absorption enhancing ingredient may by included with a therapeutic agent intended for systemic administration to enhance the transport of the therapeutic agent through the bladder wall to the plasma. References to therapeutic agents herein are intended to include one or more therapeutic agents as well as such therapeutic agent formulations, unless the context indicates otherwise.

The amount of pharmaceutically-active agent present depends upon the particular pharmaceutically-active agent employed and the medical condition being treated. In one embodiment of the invention, the pharmaceutically-active agent or microspheres containing pharmaceutically-active agent are present in an effective amount. In another embodiment, the amount of pharmaceutically-active agent or microspheres containing pharmaceutically-active agent represent from about 0.01% to about 60% of the coating by weight, based upon the total weight of the coating. In another embodiment, the amount of pharmaceutically-active agent or microspheres containing pharmaceutically-active agent represents from about 0.01% to about 40% of the coating by weight, based upon the total weight of the coating. In another embodiment, the amount of pharmaceutically-active agent or microspheres containing pharmaceutically-active agent represent from about 0.1% to about 20% of the coating by weight, based upon the total weight of the coating.

Any suitable weight proportion of therapeutic agent may be used, based upon the weight of the implant exclusive of the therapeutic agent. The proportion of therapeutic agent to non therapeutic agent implant material may vary, for example, from about 0.01 to about 40 percent by weight, preferably from about 0.1 to about 10, based upon the total weight of the implant.

The hydrophilic foam coating can bear any one or more of a variety of therapeutically useful agents, for example, agents that can aid in the healing of bladder 10 and the reduction of urgency, such as oxybutynin, or other anticholinergic agents. Furthermore the hydrophilic foam, or other drug immobilizing means, can be used to carry genetic therapies, e.g., for replacement of missing enzymes, to treat cystopathies at a local level, and to release palliative agents. More broadly, a useful therapeutic agent can be any compound that is biologically active and requires short or long term administration to a tissue or organ for maximum efficacy. Therapeutic agents that can be used in accordance with the present invention include, but are not limited to, antibiotics, antimuscarinic agents, anticholinergic agents, antispasmodic agents, calcium antagonist agents, potassium channel openers, musculotropic relaxants, antineoplastic agents, polysynaptic inhibitors, and beta-adrenergic stimulators. Examples of anticholinergic agents are propantheline bromide, imipramine, mepenzolate bromide, isopropamide iodide, clidinium bromide, anisotropine methyl bromide, scopolamine hydrochloride, and their derivatives. Examples of antimuscarinic agents include, but are not limited to, hyoscyamine sulfate, atropine, methantheline bromide, emepronium bromide, anisotropine methyl bromide, and their derivatives. Examples of polysynaptic inhibitors include baclofen and its derivatives. Examples of .beta.-adrenergic stimulators include terbutaline and its derivatives. Examples of calcium antagonists include terodiline and its derivatives. Examples of musculotropic relaxants include, but are not limited to, dicyclomine hydrochloride, flavoxate hydrochloride, papaverine hydrochloride, oxybutynin chloride, and their derivatives. Examples of an antineoplastic agents include, but are not limited to, carmustine levamisole hydrochloride, flutamide, (w-methyl-N-[4-nitro-3-(trifluoromethyl)phenyl]), adriamycin, doxorubicin hydrochloride, idamycin, fluorouracil, cytoxan, mutamycin, mustargen and leucovorin calcium. Examples of antispasmodic agents are hexadiphane, magnesium gluconate, oktaverine, alibendon, butamiverine, hexahydroadiphene, 2-piperidinoethyl 3-methylflavone-8-carboxylate, 4-methylumbelliferone 0,0-diethyl phosphorothiate. Examples of potassium channel openers include pinacidil and N-[-2-Nitrooxy)ethyl]-3-pyridinecarboxamide.

Additionally, a potential significant use of the therapeutic agent delivery implant is as a delivery system for chemotherapeutic agents to treat bladder cancer. The therapeutic agent delivery implant of the present invention, can with a, single application, deliver a sustained dose of chemotherapeutics to the mucous membrane of the bladder 10 for periods of up to about 28 days, at which time an implant such as implant 42 can be replaced with a fresh therapeutic agent-laden implant, if desired. By delivering the therapeutic agent continuously to the tumor, more of the tumor cells can be exposed to the therapeutic agent during their proliferative phase when they are most sensitive to the chemotherapy. Additionally, the dose of the therapeutic agents can be kept lower then in the usual interrupted, short-term treatment, thus minimizing irritation and discomfort to the patient. Further, the fact that one minor procedure is needed for insertion and one for removal provides less inconvenience to the patient and better cost efficiency then with the usual interrupted, short-term treatment.

Therapeutic agents that do not readily cross to the plasma barrier offered by the wall of the urothelium may be employed for local usage, for example, to treat bladder-related conditions, while therapeutic agents that readily cross to the plasma barrier may be systemically administered via bladder implantation of the implants. Some therapeutic agents may have dual functionality, being locally useful and also being systemically absorbable.

The therapeutic agent delivery implant can be useful in the delivery of antibiotics to the urinary tract, and especially bladder 10. The present invention provides methods of treating such cases comprising implantation of a therapeutic agent delivery implant, such as implant 42, containing an antibiotic which is inserted into bladder 10 as a prophylactic measure to preempt possible urinary tract infection. The therapeutic agent delivery implant can be replaced on a regular basis, in one embodiment of the present invention approximately monthly or every twenty-eight days. Other replacement periods may be employed, if desired, for example, from about 7 days to about two months, more preferably from about two to about six weeks. Toward two months problems arising from encrustation and the like may be expected.

Further, the therapeutic agent delivery implant can deliver antibiotics for the treatment of systemic chronic infections. For example, diseases such as Lyme Disease, tuberculosis, or even periodontitis require the long-term administration of antibiotics, sometimes for as long as six months to years. Some diseases also require the long-term treatment using intravenous antibiotics requiring doctor visits or skilled nursing care. Often a special catheter needs to be surgically inserted into a vein or under the skin. The inventive implants can be inserted into the bladder 10 and may be changed about once a month under a local anesthetic greatly ameliorating these problems.

Some other therapeutic agents that may be delivered to the bladder include antispasmodics to treat overactive or spastic bladders with desensitizing or antispasmodic agents. Overactive bladder and spastic bladder conditions area significant problem, and the possibility of placing an implant such as domical implant 42 in the bladder that does not impinge on the bladder neck (the dome-shaped implant) while allowing the chronic delivery of a desensitizing agent for comfort or an antispasmodic agent is another benefit of the invention.

In addition, systemically acting therapeutic agents may be delivered by the implants of the invention. There are many therapeutic agents that require injection on a regular basis, for example, growth hormone. Proteins of which growth hormones are exemplary are fragile and cannot be taken orally due to destruction in the stomach due to the action of stomach acid and of proteolytic enzymes. Accordingly, they are delivered by daily injection. Such daily injections can be entirely avoided or reduced by delivering such labile therapeutic agents through the bladder mucus membrane employing the implants of the present invention.

In use, the health care provider can, if desired, determine the size of the desired therapeutic agent delivery implant according to the invention by imaging the bladder, such as by radiography or ultrasound. Optionally, an implant can come in a single size that expands to fit a bladder. An implant would have at least one desired biologically active compound added either at the site of insertion, or come prepackaged with the compound or compounds. The implant would then be compressed and loaded into a tubular structure, such as a trocar, cannula, fiberoptic cannula, catheter, or minimally invasive rigid or flexible scope, such as a cystoscope, or the like. Alternatively, the implant can come prepackaged in a tube that fits into an insertion device, such as a cystoscope. The insertion device would then be threaded up the urethra 30 into bladder 10, optionally with the use of a topical anesthetic. Once in bladder 10, the implant would then be released from the tubular structure into a bladder 10, preferably away from the trigone 22. In one method, the implant would be released into the dome of bladder 10, away from the trigone to prevent undesirable reactions at the trigone and keep the implant away from the ureters. Once the therapeutic agent is used up, the implant can be removed, usually after a cycle of from about 2 to 8, preferably about 4, weeks to prevent any risk of an immune response to the foreign object. Additionally, if long term treatment with successive application of therapeutic agent delivery implants is desired, the spent therapeutic agent delivery implant should be removed when the fresh therapeutic agent delivery implant is implanted to avoid adversely impacting the urine retaining capacity of bladder 10, and other potential problems.

If removal is necessary, a removal instrument such as a trocar, cannula, fiberoptic cannula, catheter, or minimally invasive rigid or flexible scope, such as a cystoscope, or the like, can be inserted into the bladder 10, used to grip a portion of the implant which may then be pulled into the removal instrument, thus compressing the implant for removal. The removal instrument can have a hook, grasping forceps or other similar device that grabs a piece of the therapeutic agent delivery implant.

Alternatively, implant 70 can have attached to it a cord 72 which extends externally from the urethra, as shown in FIG. 4, then, when the biologically active substance is exhausted, cord 72 can be pulled into the cystoscope enabling implant 70 to be drawn into the instrument and compressed for removal through the urethra.

Some of the above-described benefits, and others, of the novel hemispherical or domical implants and implant systems, such as implant 42, that are provided by the invention can be summarized as follows:

Large volume. By constructing the implant as the outermost layer of a solid object approximating the shape and size of the available volume at the implantation site, a relatively large volume of implant of resilient porous material can be inserted to a mammalian body site such as bladder 10, where implant 42 can, if desired, extend around the entire superior portion of bladder 10. Such an implant may be deployed within the bladder and rest in the dome of the bladder, located in place by the outward elastic compressibility of the foam, preventing the implant from being dislodged and intruding on the bladder neck 28 and trigone 22. Its large volume enhances capacity of the implant to bear biological actives.

Large surface area. The hollow hemispherical or related configuration of implant 42 provides a very substantial internal surface area for diffusion of drugs and a large external surface area to permit access to, and enhance flow of ambient fluids, for example urine, to the therapeutic agent-bearing internal surfaces.

By locating itself adjacent the walls of the implantation site, for example, bladder inner walls 12, implant 42 presents a large surface area in close proximity to or even in contact with the bladder mucosa for ready transfer of therapeutic agents to the bladder mucosa for systemic absorption.

Simple shape. A hemisphere, or dome, is a simple shape easily fabricated.

Compressible. A hemisphere, or dome, rendered in low bulk density reticulated resilient foam lends itself to being compressed and loaded within an introducer cannula, cystoscope or the like.

Obstruction-free. Even if the foam material were to cover ureter openings 25, 27, the openings would not be obstructed because the porous material of the implant can permit urine flow.

Self-locating. A particularly significant benefit of implant 42 is a natural ability of the domical shape of the implant to remain stable and in place against the dome of the bladder, preventing the implant form floating freely in the contents of the bladder and avoiding contact with the trigone 22.

Durability. Employment of a foam composite implant material having a durable hydrophobic polyurethane, or possibly a polycarbonate scaffold provides an implant that can be inserted for extended periods, e.g., up to 28 days, if desired, without degrading into fragments or particles that could cause blockage of functional biological structures such as ureters 25, 27 or urethra 30. In some cases, longer or even permanent implantation may be possible.

High dosage. Shell-like implants such as domical implant are advantageous in being able to release therapeutic agents or other active agents at a relatively high dosage, albeit for a relatively short period of time. The large external surface area and thin wall construction promote flow-through and drug elution.

Therapeutic Agent Packaging

The therapeutic agents to be delivered by the implants and methods of the invention may be suitably packaged, for example, to have desired release characteristics, for secural to the implants of the invention. Advantageously, such packaging may comprise degradable microspheres or microcapsules.

One such controlled release formulation comprises biodegradable polymer microspheres containing an biologic active agent which microspheres are secured to a hydrophobic foam scaffold for example, by adhesive or by being retained in a layer of hydrophilic polymer matrix, e.g., a hydrophilic polyurethane foam coating the scaffold pores.

The hydrophilic polyurethane layer can act as both a reservoir and a carrier. The carrier immobilizes the microspheres and allows the microspheres to release a material at a controlled rate or rates or at a controlled release time or times at a specific site. By avoiding use of a conventional adhesive, the release kinetics can be enhanced, by avoiding interference from the degradation of an adhesive. The microspheres can comprise a solution, solid gel or other formulation of the biologic agent contained in a semipermeable housing with controlled water permeability.

Introducer Instruments

Various introducer instruments, for example, cannulae, trocars, catheters, or minimally invasive rigid or flexible instruments, optionally one incorporating visualization or electromechanics, such as a cystoscope, laproscope, arthroscope, or endoscope, or the like, may be employed to introduce the implants of the invention to desired mammalian corporeal sites, and to remove the implants, if desired, as will be apparent to those skilled in the art. Some suitable instruments are illustrated, by way of example, in FIGS. 16-20.

The introducer instrument shown in FIG. 16, that is, rigid cystoscope 200, comprises a body 202 having a hand grip 204 for manipulating the cystoscope and a hollow shaft 206 extending from body 202. The proximal end 208 of the cystoscope body 202 is provided with portals (not shown) to receive various instruments, for example, a catheter or forceps, or connections for services such as suction, gas and/or water, as well as a viewing portal. Older cystoscopes employ telescope-like optical arrangements for viewing the work site, but more recent cystoscopes employ fiber optics and output an image to a video monitor. The tip 210 of cystoscope 200 generally contains portals through which the various instruments or services employed, as well as viewing devices such as fiber optics, may be passed.

Hollow shaft 206 of cystoscope 200, as shown in FIG. 16, is substantially rigid so that the relevant anatomy has to be substantially aligned for hollow shaft 206 to be inserted through the urethra to deliver an implant to the bladder.

The flexible cystoscope 220 shown in FIG. 17 has similar components to the rigid cystoscope of FIG. 16, as indicated by use of the same reference numerals, with the difference of a flexible hollow shaft 222 in place of rigid shaft 206 and an optional winder mechanism 224. Winder mechanism 224 can be rotated to move the distal tip 226 of flexible shaft 222 from side to side. Cystoscopes employing other mechanisms, for example, joystick-like controls actuating miniature motors enabling a variety of movements of the cystoscope tip 226 may also be used.

Cystoscope 200 can be employed to deliver an implant to the bladder by inserting flexible shaft 222 into the urethra, without requiring anatomical alignment.

The catheters illustrated in FIGS. 18 and 19 are two examples of forceps-equipped catheters that can be employed for implantation and retrieval of implants in accordance with the present invention. As shown in FIG. 18, catheter 230 comprises, at its proximal end, a scissor-like actuation mechanism 232, a hollow shaft 234 that contains a linkage 236, and an end tool, in this case forceps 238 that can be projected through the distal end 240 of catheter 230. Forceps 238 are shown in a retracted, closed position where they can grip an implant (not shown) within catheter 230.

Actuation mechanism 232 comprises a pair of scissor blades 242, 244, pivoted together at 246, of which blade 242 is attached to catheter shaft 234 and blade 244 is attached to linkage 236. Each blade 242, 244 bears a handle 248, 250 respectively. Manipulation of handles 248, 250 operates through linkage 236 to actuate forceps 238 which can grasp and release an implant such as implants 42, 80, 90, 94, 96, 100, 110 or 120, to draw the implant into catheter 230 and expel it therefrom for insertion at, or removal from, a particular body site.

The catheter 260 illustrated in FIG. 19 has a modified, syringe-style actuation mechanism 262 comprising a plunger 264 and finger rests 266 and 268 either side of plunger 264. Parts with the same reference numerals are similar to those of catheter 230, as shown in FIG. 18. Forceps 238 is shown in an advanced, opened position after releasing an implant or preparatory to grasping an implant.

As shown in to FIG. 20, a modified end mechanism for a catheter such as catheter 230 shown in FIG. 18, comprises a sleeve 270 inserted into a catheter end 272. An implant such as implant 90, in compressed configuration, can be contained within sleeve 270 in catheter end 272. Implant 90 can be compressed and assembled into sleeve 270 prior to insertion into catheter end 272 and could be supplied in this form by a vendor, facilitating the medical practitioner's procedure. Catheter end 272 has an inwardly facing peripheral retainer lip 274 that can engage and retain sleeve 270 so that when the end mechanism is actuated, implant 90 is expelled from the catheter and sleeve 270 remains within the catheter.

Treatment Methods of the Invention

The invention also provides treatment methods utilizing the novel implants described herein which may be utilized in combination with suitable introducer instruments, as described hereinabove. The combination of an expansible implant, as described herein, bearing a biological active to be delivered in situ, and retained in an introducer apparatus in compressed configuration provides a novel implantation apparatus useful for a variety of treatments of mammals, especially humans, according to the nature of the biological active.

Thus, the invention provides a treatment method comprising inserting an introducer instrument, charged with one or more implants in compressed configuration and bearing one or more biological agents, each as described herein, into a mammalian corporeal site, for example the human bladder, and releasing the implant or implant at the corporeal site. The implant expands at the site, opening up its pores or interstices to passage of ambient body fluids, e.g., urine which can elute the one or more biological agents from the implant for local or systemic use. If necessary, protective coatings or embedding material around the biological active may be eroded away by the ambient body fluid.

The treatment methods can also optionally include any one or more of the following elements: removing the implant from the treatment site at the end of a treatment period utilizing a suitable instrument; loading the implant into an introducer instrument; compressing the implant; and manipulating the implant in situ to a desired position, orientation or configuration employing a suitable instrument; as well as imaging the implantation site and selecting a suitable implant according to the characteristics of the site image.

Therapeutic Compositions

The invention also provides a range of novel therapeutic compositions that can be effected employing the novel implants of the invention. A simple composition comprises an effective quantity, for the intended implantation period and therapy, of a primary biologic agent intended to treat a condition. The quantity can be varied according to whether the biologic agent is to be utilized locally, e.g., in the bladder, or systemically after transmission across the bladder mucosa to the plasma. Any suitable and effective quantity can be supported on one or more implants to constitute an individual treatment.

In one embodiment of the invention, the quantity corresponds with the quantity required to provide a desired average local concentration of the particular biologic agent, in accordance with its known efficacy, within the bladder, or other site, for the intended period of implantation, e.g., 7, 14, 28, or 42 days.

In another embodiment of the invention, the quantity corresponds with the quantity required to provide a desired concentration of the particular therapeutic agent, in accordance with its known efficacy, in the bloodstream for the intended period of implantation, e.g., 7, 14, 28, or 42 days. In either case, due allowance can be made for losses due to urination, for example from ten to fifty percent loss allowance could be made depending upon the individual patient and their routines.

In addition to a primary therapeutic agent, or therapeutic agent intended to treat a condition, for example, infection or tumor growth, one or more auxiliary therapeutic agents may be included in the therapeutic composition. Such auxiliary therapeutic agents can perform one or more of various supplemental roles. For example, one such role is tolerance enhancement to enhance the patients tolerance of the implant system. Another useful role is membrane permeability enhancement, or membrane solubilization, to facilitate transport of the primary therapeutic agent across the bladder mucosa to the plasma for systemic utilization or delivery of the primary therapeutic agent. Other useful roles and therapeutic agents or other agents that may fulfil them and be employed in the therapeutic compositions of the invention will be apparent to those skilled in the art.

Therapeutic agents or pharmaceuticals useful for tolerance enhancement are intended to modulate the responses of local biologic structures in the vicinity of the implant to the presence of the implant, or to contact with the implant or of responses to implant elutants to reduce undesired micturition, urination or incontinence or to ameliorate discomfort, irritation or pain.

Some useful such a therapeutic agents include, by way of example, antispasmodic drugs, for example, oxybutinin, and agents affecting the afferent nerves for mechano-receptors, specifically the c-fiber afferents and agents which block the vanilloid receptor subtype 1(VR1). A representative therapeutic agent having such capability is capsaicin which can be employed at an effective dosage, as described in connection with the primary agent, for example, a dose in the range to provide concentrations within the bladder of from about 1 mg/kg to about 45 mg/kg or from about 0.1 mM to about 10 mM. Another useful drug is resiniferatoxin, which affects dorsal root mechano-chemo receptors in a desensitization manner. A suitable dosage range is from about 1 nM to 1000 nM, preferably from about 10 nM to about 100 nM to provide a desired slow release.

Where a the primary therapeutic agent is intended to be delivered to the bloodstream via the bladder mucosa, a membrane permeability enhancing agent can be included in the therapeutic composition in quantities or concentrations to provide an effective concentration at the relevant membrane. Some examples of suitable agents for use in the bladder are protamine sulfate and polypropylene glycol.

Among the primary therapeutic agents that may be employed include genetic agents, preferably nonviral genetic therapy agents that can modify local cells, for example, bladder wall cells to provide useful results such as the local production of insulin for the treatment of inherited juvenile diabetes. Genetic therapy may also be provided for other hormones or factors regarding which the patient is deficient. A membrane permeation enhancer, or solubilizer is desirably also included to deliver the genetic therapy agent to the basal or intermediate cells.

As shown in FIG. 16, the innermost layer of the bladder wall 12, urothelium 32, as described above comprises a basal cell layer 280, an intermediate cell layer 282 and an innermost layer 284 of epithelial umbrella cells 286. The luminal surfaces of the umbrella cells 286 are coated with a layer 288 of glycosaminoglycans and

The basal cell layer 280 is separated from the connective tissue and elastic fibers of submucous coat 34 by a basal lamina 290. The glycosaminoglycan-coated surfaces of the umbrella cells 286 line the bladder inner walls 12 and accordingly interface with urine in the bladder. Thus, the permeability of this coated layer largely determines whether a given substance can be systemically absorbed from the bladder and can be enhanced by therapeutic agents such as protamine sulfate or polypropylene glycol, as described herein.

One preferred mechanism of genetic therapy comprises modification of the intermediate cells 282 to cause them to generate insulin or another therapeutic agent. The generated insulin or the like moves toward the basal cell layer 280 and be absorbed into the blood stream. The interstices in the intermediate cell layer 282 can provide sites for the accumulation of such locally generated agents or agents released from an inventive implant in the bladder and transported across the bladder mucosa whence they may be steadily absorbed into the bloodstream.

Some literature of interest in connection with delivery of agents via the bladder includes: Fraser et al., “The Future of Bladder Control-Intravesical Drug Delivery, a Pinch of Pepper, and Gene Therapy” Reviews in Urology vol. 4, no. 1 (2002); Szallasi, A., et al., AResiniferatoxin-type phorboid vanilloids display capsaicin-like selectivity at native vanilloid receptors on rat DRG neurons and at the cloned vanilloid receptor VR1., 1999., 128(2):, 428-434.; Macha, A., et al.; “APhorboid 20-homovanillates induce apoptosis through a VR1-independent mechanism.”, Chem. Biol., 2000, 7(7):, 483-492. Szallasi, A., & P. M. Blumberg. and AVanilloid (Capsaicin) receptors and mechanisms, Pharmacol. Rev., 1999, 51:, 159.

The individual therapeutic agent or composition may be absorbed on the implant. Alternatively, the therapeutic composition or the biologic agent may be chemically bound to the implant, or one or more components of the composition may be chemically bonded and another or others may be chemically bound. A preferred means of immobilizing the therapeutic composition is by micropackaging, for example, in microspheres, as described elsewhere herein.

Other Aspects of the Invention

In other aspects the present invention provides device-therapeutic agent therapy for urinary tract infections employing bioactive polymeric materials and novel polymeric matrices such as the implants described herein, which materials are also useful in endovascular applications addressing cardiovascular, neurological, and peripheral vascular conditions. Furthermore, the invention provides biosystems applications employing such polymeric materials and matrices and which involve the immobilization and controlled release of biologics for a range of clinical purposes, including, for example, without limitation, use of such polymeric foam composites for non-active wound care applications.

Pursuant to the present invention such biosystems applications can employ a polyurethane foam composite based on a combination of a reticulated hydrophobic polyurethane scaffold and a hydrophilic polyurethane coating, some examples of which are disclosed in Thomson, supra. A valuable functionality of the composite foam includes an ability to immobilize and release therapeutic agents, high flow-through characteristics, biocompatibility and immunogenicity. Tests with collagenase demonstrate an ability to immobilize and retain enzymatic activity within the polymer system. In addition, the composite foam material can support mammalian, especially but not exclusively human, cell propagation and proliferation into the polymeric matrix and the present invention includes cell proliferation or propagation systems realized in the polymeric matrices disclosed herein and in the references incorporated herein.

The invention also includes endovascular applications of the encompassing implantable polymeric devices delivered into the vascular system for interventional neuroradiological, interventional cardiological peripheral vascular and other purposes.

Biosystems applications according to the invention can encompass extracorporeal and short-term (less than 28 days) implantable polymeric devices delivered into the gastrointestinal or intravesicular cavities for the controlled release and/or sustained activity of therapeutic agents, including enzymes, especially enzymes employed for enzyme therapy of urinary tract infections.

While reference has been made herein to mammals, it will be understood that the inventive implants can be employed to treat other animal classes such for example, as birds, reptiles or the like. Particular mammals of interest are primarily humans but also commercially valuable species such as horses, pigs, cattle, sheep, other primates, dogs and cats, and the like, as well as laboratory animals such as mice and rats.

As an example of certain aspects of the invention, the results of represnetative testing are set forth below:

### EXAMPLE 1

A reticulated TDI/polyether based foam (SIF grade obtained from Foamex) was used as a scaffold or substrate. The foam substrate was 4″×4″ (10.2 cm×10.2 cm) square sample with a thickness of ¼″ or 0.635 cm with a volume of 65.5 cm³ and weighed 1.2 g, giving a density of 0.0183 gms/cc. 2.42 grams of hydrophilic polyurethane prepolymer (Urepol 1002A obtained from Envirochem) and 64 milligrams of Thio-TEPA was diluted in 6 ml of DMF. The foam was dipped into the solution mixture of polyurethane prepolymer, Thio-TEPA and DMF. The Thio-TEPA containing solution mixture of polyurethane prepolymer and DMF was then distributed and spread over the foam substrate. During the spreading and distribution, it was ensured that solution mixture contacted the foam substrate from all sides and coated both the surface and internal pores of the foam substrate.

The foam substrate with coated solution mixture was held overnight below 8° C. The coating was cured by the reaction of the ambient moisture with the available reactive isocyanate in the prepolymer. After the overnight curing, the foam substrate with coated solution mixture was vacuum-dried to remove residual solvents to leave a coating of polyurethane containing Thio-TEPA.

The final weight of the foam substrate and the drug loaded coating was 3.24 gms. Density of the coated foam substrate was 0.0495 gms/cc. The drug loading was 0.009 gms per 1 gm of coated foam substrate, i.e. 0.9 wt %. The drug entrapment efficiency was 50% calculated from a final measures drug loading of 0.9 wt % and a theoretical drug loading of 1.8 wt %. The weight of the drug in the final coated foam substrate was 0.029 gms or 29 mgs with the drug density on the coated foam substrate being 0.44 mgs per cc.

Conditions used in the preliminary coating experiment along with drug loading results are presented in the following table:

TABLE 1

| | | Hydrophilic Prepolymer | | | | | | | |
|---|---|---|---|---|---|---|---|---|---|
| | | Solution System | | | Pre- | Post- | Theor. | Final | |
| | | | Solvent | Drug | coating | coating | drug | drug | Entrapment |
| | | Prepolymer | added, | weight, | Foam | Sample | loading, | loading, | efficiency, |
| Drug | Lot Number | weight, g | mL | mg | wt, g | wt, g | wt % | wt % | % |
| Thio-TEPA | J1168-027-1 | 2.42 | 6 | 64 | 1.2 | 3.24 | 1.8 | 0.9 | 50 |

The coated foam substrate was placed in phosphate buffer at 37 C, and a pH of 7.4 and the Thio-TEPA in vitro release was measured. The results are presented in the table below:

TABLE 2

| | | Core | Cumulative Release, | | |
|---|---|---|---|---|---|
| | | loading, | % at Day | | |
| Lot No: | Drug | wt % | 1 | 3 | 6 |
| J1168-027-1 | Thio-Tepa | 0.9 | 11.9 | 43.7 | 66.6 |

The polyurethane coated polyurethane foam substrate successfully demonstrated its ability release the Thio-Tepa over a period of time or show controlled release capabilities.

### EXAMPLE 2

A cast film of polyurethane containing Ciprofloxacin was made by reacting hydrophilic polyurethane prepolymer (Urepol 1002A obtained from Envirochem) with distilled water. The reacting mixture was spread in a thin film over a glass petri dish. The film was cured overnight and dried to leave a thin film of polyurethane containing Ciprofloxacin. The resultant drug carrying film was further vacuum-dried. The drug loading was measured to be 0.06 gms per 1 gm of coated film, i.e. 6.0 wt %.

The drug carrying film was placed in phosphate buffer at 37 C and a pH of 7.4 and the Ciprofloxacin in vitro release was measured. The results are presented in Table below:

| | | Core | Cumulative Release, | | |
|---|---|---|---|---|---|
| | | loading, | % at Day | | |
| Lot No: | Drug | wt % | 1 | 3 | 4 |
| J1168-018-7 | Ciprofloxacin | 6.0 | 15.7 | 16.8 | 29.28 |

The polyurethane film successfully demonstrated its ability release Ciprofloxacin over a period of time or show controlled release capabilities.

The entire disclosure of each patent and patent application cross-referenced or referenced herein and of each non-patent publication referenced herein is hereby incorporated herein by reference thereto, as though wholly set forth herein. Each document incorporated by reference in any of the foregoing patents, patent applications or non-patent publications is also incorporated herein in its entirety by reference thereto.

While illustrative embodiments of the invention have been described above, it is, of course, understood that many and various modifications will be apparent to those of ordinary skill in the relevant art, or may become apparent as the art develops. Such modifications are contemplated as being within the spirit and scope of the invention or inventions disclosed in this specification.

We claim:

1. A therapeutic agent delivery implant for implantation into a patient's body, said implant comprising:
a resilient or flexible, at least partially hydrophobic reticulated elastomeric support scaffold and
one or more therapeutic agents secured to and/or supported by the scaffold for release within the patient.

2. A therapeutic agent delivery implant for implantation into a patient's body, said implant comprising:
a resilient or flexible, at least partially hydrophobic reticulated elastomeric support scaffold; and
a hydrophilic coating arranged on said scaffold,
wherein said coating contains one or more therapeutic agents for release within the patient.

3. The implant of claim 2, wherein at least one therapeutic agent is secured to and/or supported by the scaffold.

4. The implant of claim 1 or 2, wherein at least one therapeutic agent is contained within microspheres.

5. The implant of claim 1 or 2, wherein the scaffold is biodurable.

6. The implant of claim 2, wherein at least one therapeutic agent is contained within microspheres in the coating.

7. The implant of claim 2, wherein the coating contains enzymes.

8. The implant of claim 1 or 2, wherein the the scaffold comprises a hydrophobic polyurethane.

9. The implant of claim 2, wherein the coating comprises a hydrophilic polyurethane.

10. The implant of claim 1 or 2, wherein the therapeutic agent is selected from the group consisting of a pharmaceutical, a growth factor, an enzyme, RNA, DNA, a nucleic acid, and a vector, and mixtures of two or more thereof.

11. The implant of claim 1 or 2 which has a hemispherical, bullet, football, cylindrical, spherical, or irregular shape.

12. The implant of claim 11 which is spaghetti-shaped.

13. A method of delivering an implant to a mammalian site, which comprises the steps of:
(a) collapsing and/or compressing an implant of claim 1 or 2;
(b) inserting the implant from step (a) into a rigid or flexible delivery instrument having a distal tip;
(c) advancing the delivery instrument distal tip to a desired site;
(d) deploying the implant at the desired site, whereby the implant expands to a size and shape substantially similar to its size and shape before step (a); and
(e) withdrawing the delivery instrument.

14. The method of claim 13, wherein the delivery instrument is a cannular, trocar, catheter, or a minimally invasive rigid or flexible instrument.

15. The method of claim 14, wherein the minimally invasive instrument incorporates visualization or electromechanics.

16. The method of claim 15, wherein the minimally invasive instrument has a fiberoptic guide.

17. The method of claim 14, wherein the minimally invasive instrument is a cystoscope, laproscope, arthroscope, or endoscope.

18. The method of claim 13, wherein the desired delivery site is the patient's bladder and the delivery instrument is advanced through the patient's urethra.

19. A method of treating a patient, which comprises the steps of:
(a) collapsing and/or compressing an implant of claim 1 or 2;
(b) inserting the implant from step (a) into a rigid or flexible delivery instrument having a distal tip;
(c) advancing the delivery instrument distal tip to a desired site;
(d) deploying the implant at the desired site whereby the implant expands to a size and shape substantially similar to its size and shape before step (a);
(e) withdrawing the delivery instrument; and
(f) leaving the implant in place for a desired period of time.

20. The method of claim 19, which also comprises the steps of:
(g) advancing the distal tip of a removal instrument to the desired site;
(h) engaging the implant; and
(i) withdrawing the implant and the removal instrument from the patient.

21. The method of claim 19, wherein the delivery instrument is a cannular, trocar, catheter, or a minimally invasive rigid or flexible instrument.

22. The method of claim 21, wherein the minimally invasive instrument incorporates visualization or electromechanics.

23. The method of claim 22, wherein the minimally invasive instrument has a fiberoptic guide.

24. The method of claim 21, wherein the minimally invasive instrument is a cystoscope, laproscope, arthroscope, or endoscope.

25. The method of claim 20, wherein the removal instrument is a cannular, trocar, catheter, or a minimally invasive rigid or flexible instrument.

26. The method of claim 25, wherein the minimally invasive instrument incorporates visualization or electromechanics.

27. The method of claim 26, wherein the minimally invasive instrument has a fiberoptic guide.

28. The method of claim 25, wherein the minimally invasive instrument is a cystoscope, laproscope, arthroscope, or endoscope.

29. A method of systemically or locally treating a patient, which comprises the steps of:
(a) positioning an implant of claim 1 or 2 at a desired site within a patient; and
(b) leaving the implant at the desired site for a suitable period of time.

30. A system for treating a patient, which comprises an implant of claim 1 or 2 and a delivery instrument.

31. The system of claim 30, wherein the delivery instrument is a cannular, trocar, catheter, or a minimally invasive rigid or flexible instrument.

32. The system of claim 31, wherein the minimally invasive instrument incorporates visualization or electromechanics.

33. The system of claim 32, wherein the minimally invasive instrument has a fiberoptic guide.

34. The system of claim 31, wherein the minimally invasive instrument is a cystoscope, laproscope, arthroscope, or endoscope.

35. The system of claim 30 which also comprises a removal instrument.

36. The system of claim 35, wherein the removal instrument is a cannular, trocar, catheter, or a minimally invasive rigid or flexible instrument.

37. The system of claim 36, wherein the minimally invasive instrument incorporates visualization or electromechanics.

38. The system of claim 37, wherein the minimally invasive instrument has a fiberoptic guide.

39. The method of claim 36, wherein the minimally invasive instrument is a cystoscope, laproscope, arthroscope, or endoscope.

40. A method of treating a local urological condition in a patient, which comprises the steps of:
(a) collapsing and/or compressing an implant of claim 1 or 2;
(b) inserting the implant from step (a) into a rigid or flexible delivery instrument having a distal tip;
(c) advancing the delivery instrument distal tip through the patient's urethra to the bladder;
(d) deploying the implant in the bladder whereby the implant expands to a size and shape substantially similar to its size and shape before step (a);
(e) withdrawing the delivery instrument; and
(f) leaving the implant in place in the bladder for a desired period of time.

41. The method of claim 40, wherein the local condition to be treated is cancer, an infection, an inflammation, a neurological condition, or a trauma,

42. A method of treating a condition in a patient that is systemic or external to the bladder, which comprises the steps of:
(a) collapsing and/or compressing an implant of claim 1 or 2, wherein the implant or the coating thereon comprises a solubilizer;
(b) inserting the implant from step (a) into a rigid or flexible delivery instrument having a distal tip;
(c) advancing the delivery instrument distal tip through the patient's urethra to the bladder;
(d) deploying the implant in the bladder whereby the implant expands to a size and shape substantially similar to its size and shape before step (a); (e) withdrawing the delivery instrument; and
(e) leaving the implant in place in the bladder for a desired period of time.

43. The method of claim 42, wherein the condition to be treated is cancer, an infection, an inflammation, a neurological condition, or osteomylitis.

44. A therapeutic agent delivery implant for implantation to a mammalian site, the implant comprising:
a resilient or flexible,hydrophobic support reticulated elastomeric scaffold and
at least one therapeutic agent secured to and supported by the scaffold for release at the mammalian site,
wherein the therapeutic agent delivery implant is insertable into a mammalian bladder or other suitable site via the urethra and is locatable within the bladder.

45. The implant of claim 44, wherein the implant is capable of being kept out of stimulative contact with the trigone during the normal daily host routine.

46. The implant of claim 44, wherein the therapeutic agent delivery implant remains stable and fixed against the mucous membrane of the bladder away from the trigone.

47. The implant of claim 44, wherein the therapeutic agent delivery implant is locatable in the dome of the bladder and permits flow of urine through the therapeutic agent delivery implant material.

48. The implant of claim 44, wherein the therapeutic agent delivery implant is shaped to engage and lodge against the bladder inner wall.

49. The implant of claim 44, wherein the therapeutic agent delivery implant is configured, sized and prestressed to have a cross-sectional area in excess of the anticipated maximum cross-sectional area of the intended recipient bladder.

50. The implant of claim 44, wherein the therapeutic agent delivery implant is elastically compressible.

51. A method of delivering an implant to a mammalian site comprising the steps of:
(a) collapsing and/or loading into a delivery instrument a resiliently compressible therapeutic agent delivery implant having an expanded configuration when deployed;
(b) advancing the delivery instrument through a mammalian urethra to access the bladder;
(c) deploying the therapeutic agent delivery implant through the delivery instrument into the bladder; and
(d) withdrawing the delivery instrument, leaving the therapeatic agent delivery implant in the bladder.

52. The method of claim 51, wherein the therapeutic agent delivery implant can be pulled into a removal instrument, insertable into the urethra, and the method further comprising the steps of:
(e) advancing the removal instrument into the urethra and
(f) removing the therapeutic agent delivery implant from the bladder with the removal instrument.

53. The method of claim 51 or 52, wherein the delivery instrument and the removal instrument are each a cannular, trocar, catheter, or a minimally invasive rigid or flexible instrument.

54. The method of claim 53, wherein the minimally invasive instrument incorporates visualization or electromechanics.

55. The method of claim 54, wherein the minimally invasive instrument has a fiberoptic guide.

56. The method of claim 53, wherein the minimally invasive instrument is a cystoscope, laproscope, arthroscope, or endoscope.

57. The method of claim 52, wherein a gripping implement, deployed through the removal instrument grips the therapeutic agent delivery implant and draws it into the removal instrument.

58. The method of claim 57, wherein the gripping implement comprises a forceps or hook.

59. The method of claim 52, wherein removal is effected within from one to twenty-eight days after insertion.
